(19) United States
(12) Patent Application Publication
(10) Pub. No.: US 2025/0179509 A1
(43) Pub. Date: Jun. 5, 2025

(54) NATIVE DELIVERY OF BIOMOLECULES INTO PLANT CELLS USING IONIC COMPLEXES WITH CELL-PENETRATING PEPTIDES

(71) Applicants: BASF Plant Science Company GmbH, Ludwigshafen am Rhein (DE); RIKEN, Saitama (JP)

(72) Inventors: Joerg BAUER, Research Triangle Park, NC (US); Fang-Ming LAI, Cary, NC (US); Paul BERNASCONI, Siler City, NC (US); Marianela RODRIGUEZ, Research Triangle Park, NC (US); Vinitha CARDOZA, Research Triangle Park, NC (US); Keiji NUMATA, Saitama (JP); Boyang GUO, Saitama (JP)

(21) Appl. No.: 17/416,015

(22) PCT Filed: Dec. 20, 2019
(86) PCT No.: PCT/EP2019/086682

§ 371 Date: Jun. 18, 2021

Related U.S. Application Data
(60) Provisional application No. 62/782,816, filed on Dec. 20, 2018.

(51) Int. Cl. C12N 15/82; C12N 9/22; C12N 15/11

(52) U.S. Cl. CPC C12N 15/8206; C12N 9/22; C12N 15/11; C12N 15/8213; C12N 2310/20

(57) ABSTRACT

The invention relates to a complex comprising a first component: (i) a carrier peptide comprising a cell-penetrating sequence and a polycation sequence: and a second component (ii) a ribonucleic acid (RNA), PNA and/or protein, wherein the carrier peptide is a cyclic peptide comprising at least 2 cysteine residues bridged by a disulphide bond.

### CROSS-REFERENCE TO RELATED APPLICATIONS

This application is a National Stage Entry of PCT/EP2019/086682, filed Dec. 20, 2019, which claims priority to U.S. application No. 62/782,816, filed Dec. 20, 2018, the disclosures of each of which are hereby incorporated by reference in their entireties.

### REFERENCE TO AN ELECTRONIC SEQUENCE LISTING

The contents of the electronic sequence listing (039621-00836-Corrected-Sequence-List-ing_ST25.txt; Size: 18,207 bytes; and Date of Creation: Dec. 2, 2022) is herein incorporated by reference in its entirety.

### FIELD OF THE INVENTION

The invention is directed to methods and tools for delivering biomolecules like proteins or nucleic acids into regenerating plant cells.

### BACKGROUND OF THE INVENTION

Plant breeding is at the center of improving the agronomic performance of plants and describes processes that change the heredity of plans towards a human perceived advantage. Changes are permanent and heritable as they are reflected in the plant genome (Principles of Plant Genetics and Breeding, G. Acquaah, Wiley Blackwell 2^{nd} ed. 2012). Novel tools like gene transfer, but also improvements of the understanding of plant genomes by molecular tools (sequencing, SNP markers, pathway analysis) allow a wider application of modifications to plant genomes. Modifications are required to adapt plants to changing environmental conditions, pest pressure, stress conditions, sustainability and yield needs.

A trait of particular economic interest is increased yield. Yield is normally defined as the measur-able produce of economic value from a crop. This may be defined in terms of quantity and/or quality. Yield is directly dependent on several factors, for example, the number and size of the organs, plant architecture (for example, the number of branches), seed production, leaf senescence and more. Root development, nutrient uptake, stress tolerance and early vigor may also be important factors in determining yield. Optimizing the abovementioned factors may therefore contribute to increasing crop yield.

Seed yield is a particularly important trait, since the seeds of many plants are important for human and animal nutrition. Crops such as corn, rice, wheat, canola and soybean account for over half the total human caloric intake, whether through direct consumption of the seeds themselves or through consumption of meat products raised on processed seeds. They are also a source of sugars, oils and many kinds of metabolites used in industrial processes. Seeds contain an embryo (the source of new shoots and roots) and an endosperm (the source of nutrients for embryo growth during germination and during early growth of seedlings). The development of a seed involves many genes, and requires the transfer of metabolites from the roots, leaves and stems into the growing seed. The endosperm, in particular, assimilates the metabolic precursors of car-bohydrates, oils and proteins and synthesizes them into storage macromolecules to fill out the grain.

Plant biomass is yield for forage crops like alfalfa, silage corn and hay. Many proxies for yield have been used in grain crops. Most important amongst these are estimates of plant size. Plant size can be measured in many ways depending on species and developmental stage, but include total plant dry weight, above-ground dry weight, above-ground fresh weight, leaf area, stem volume, plant height, rosette diameter, leaf length, root length, root mass, tiller number and leaf number. Many species maintain a conservative ratio between the size of different parts of the plant at a given developmental stage. These allometric relationships are used to extrapolate from one of these measures of size to another (e.g. Tittonell et al 2005 Agric Ecosys & Environ 105:213). Plant size at an early developmental stage will typically correlate with plant size later in development. A larger plant with a greater leaf area can typically absorb more light and carbon dioxide than a smaller plant and therefore will likely gain a greater weight during the same period (Fasoula & Tollenaar 2005 Maydica 50:39). This is in addition to the potential continuation of the micro-environmental or genetic advantage that the plant had to achieve the larger size initially. There is a strong genetic component to plant size and growth rate (e.g. ter Steege et al 2005 Plant Physiology 139:1078), and so for a range of diverse genotypes plant size under one environmental condition is likely to correlate with size under another (Hittalmani et al 2003 Theoretical Applied Genetics 107:679). In this way a standard environment is used as a proxy for the diverse and dynamic environments encountered at different locations and times by crops in the field. Another important trait for many crops is early vigor. Improving early vigor is an important objective of modern rice breeding programs in both temperate and tropical rice cultivars. Long roots are important for proper soil anchorage in water-seeded rice. Where rice is sown directly into flooded fields, and where plants must emerge rapidly through water, longer shoots are associated with vigor. Where drill-seeding is practiced, longer mesocotyls and coleoptiles are important for good seedling emergence. The ability to engineer early vigor into plants would be of great importance in agriculture. For example, poor early vigor has been a limitation to the introduction of maize (Zea mays L.) hybrids based on Corn Belt germplasm in the European Atlantic.

Harvest index, the ratio of seed yield to aboveground dry weight, is relatively stable under many environmental conditions and so a robust correlation between plant size and grain yield can often be obtained (e.g. Rebetzke et al 2002 Crop Science 42:739). These processes are intrinsically linked because the majority of grain biomass is dependent on current or stored photosynthetic productivity by the leaves and stem of the plant (Gardener et al 1985 Physiology of Crop Plants. Iowa State University Press, pp68-73). Therefore, selecting for plant size, even at early stages of development, has been used as an indicator for future potential yield (e.g. Tittonell et al 2005 Agric Ecosys & Environ 105:213). When testing for the impact of genetic differences on stress tolerance, the ability to standardize soil properties, temperature, water and nutrient availability and light intensity is an intrinsic advantage of greenhouse or plant growth chamber environments compared to the field. However, artificial limitations on yield due to poor pollination due to the absence of wind or insects, or insufficient space for mature root or canopy growth, can restrict the use of these controlled environments for testing yield differences. Therefore, measurements of plant size in early development, under standardized conditions in a growth chamber or greenhouse, are standard practices to provide indication of potential genetic yield advantages.

Another trait of importance is that of improved abiotic stress tolerance. Abiotic stress is a primary cause of crop loss worldwide, reducing average yields for most major crop plants by more than 50% (Wang et al. (2003) Planta 218:1-14). Abiotic stresses may be caused by drought, salinity, extremes of temperature, chemical toxicity, excess or deficiency of nutrients (macroelements and/or microelements), radiation and oxidative stress. The ability to increase plant tolerance to abiotic stress would be of great economic advantage to farmers worldwide and would allow for the cultivation of crops during adverse conditions and in territories where cultivation of crops may not otherwise be possible.

Crop yield may therefore be increased by optimizing one of the above-mentioned factors.

There are numerous methods for modifying plant genomes, e.g. by crossing preferable alleles into the genome, selection of epigenetic changes, mutations induced by radiation or chemicals, chromosome duplications, transfer of DNA by biolistics or Agrobacterium, transient regulation using nucleic acids (e.g. RNAi) or small molecules (e.g. Salicylic acid) or regulation using different light patterns.

For many of these methods, there is a general need to deliver biomolecules into plant cells, often plant cells of a specific type (e.g. regenerating plant cells, meristematic cells, root cells etc.). Current technologies have limitations, e.g. for the delivery of proteins into plant cells. One technology used for the delivery of proteins into plant cells is biolistics. This has low success rates, high damage of cells and requires considerable experimental time and effort.

It is one objective of the invention at hand to generate plants with enhanced agronomic benefits in the fields of resistance to fungal disease, resistance to insects, germination vigor, leaf sym-metry, leaf senescence, circadian rhythm, photosynthesis regulation, meristem formation, pollen formation, pollination, seed setting, seed ripening, composition of seeds, seed size, seed number and abiotic stress tolerance such as water use efficiency, nitrogen use efficiency, phosphate use efficiency, improved UV light tolerance, improved micronutrient uptake, cold tolerance or heat tolerance.

Plants with enhanced agronomic benefits may be generated using genome editing, improving regeneration capacity, transient regulation with RNAi, ribonucleoparticle binding, protein inactiva-tion or intracellular transport regulation.

There are numerous methods for genome editing described. Beside zinc-fingers, meganucleases and TALEN, CRISPR (clustered regularly interspaced short palindromic repeats) is one methodology for precise genome editing.

The CRISPR system was initially identified as an adaptive defense mechanisms of bacteria belonging to the genus of Streptococcus (WO2007/025097). Those bacterial CRISPR systems rely on guide RNA (sgRNA) in complex with cleaving proteins to direct degradation of complementary sequences present within invading viral DNA. Cas9, the first identified protein of the CRISPR/Cas system, is a large monomeric DNA nuclease guided to a DNA target sequence adjacent to the PAM (protospacer adjacent motif) sequence motif by a complex of two noncoding RNAs: crRNA and trans-activating crRNA (tracrRNA). Later, a synthetic RNA chimera (single guide RNA or sgRNA) created by fusing crRNA with tracrRNA was shown to be equally functional (Jinek et. al. 2012).

Several research groups have found that the CRISPR cutting properties could be used to disrupt genes in almost any organism's genome with unprecedented ease (Mali P, et al (2013) Science. 339 (6121): 819-823; Cong L, et al (2013) Science 339 (6121)). Recently it became clear that providing a template for repair allowed for editing the genome with nearly any desired sequence at nearly any site, transforming CRISPR into a powerful gene editing tool (WO/2014/150624, WO/2014/204728).

Gene targeting refers to site specific gene modification by nucleic acid deletion, insertion or replacement via homologous recombination (HR). Targeting efficiency is highly promoted by a dou-ble-strand break (DSB) in the genomic target. Also, the direct presence of homology after DSB of chromosomal DNA seems to nearly eliminate non-homologous end joining (NHEJ) repair in favor of homologous recombination.

In another method, the nuclease (Cas9, Cpf1 etc.) is mutated to result only in single strand breaks (nicks) in combination with a Cytosine or Adenosine deaminase enzyme function to induce base repair (C to T, A to G) (WO15133554, U.S. Pat. No. 9,737,604). This method allows precise base editing but is limited in the bases which can be edited. For coding sequences this is of less concern (degen-eration of the genetic code), whereas for non-coding sequences the precise sequence might be of essence.

Homologous recombination (HR) as described above would allow precise base/sequence changes.

However, methods and tools are needed to improve efficiency of transfer of biomolecules, reduce cell toxicity and reduce time and cost.

### DESCRIPTION OF THE INVENTION

The invention at hand provides methods and tools to deliver biomolecules into plant cells. Surprisingly, carrier peptides have been identified comprising a cell-penetrating sequence and a pol-ycationic sequence could be identified, which enable the transport of biomolecules across plant cell walls and plasma membranes into plant cells (delivery).

Further, carrier peptides were identified which reduce the cytotoxicity in regenerating plant cells, thereby massively increase the efficiency and effectivity of the delivery of biomolecules for various methods including genome editing, targeted mutagenesis, untargeted mutagenesis, transient regulation by peptides/proteins, transient regulation by RNAi, targeted intra cellular transport of molecules and inter cellular transport of proteins/peptides in plants.

A first aspect of the invention provides a complex comprising a first component: (i) a carrier peptide comprising a cell-penetrating sequence and a polycation sequence: and a second component (ii) a ribonucleic acid (RNA), PNA and/or protein, wherein the carrier peptide is a cyclic peptide comprising at least two cysteine residues bridged by a disulphide bond.

The present inventors have identified that, surprisingly, when the carrier peptide (which comprises a cell penetrating peptide (CPP) coupled with a polycation sequence) is characterized as being a cyclic peptide comprising at least two cysteine residues bridged by a disulphide bond, this is able to bind with ribonucleic acid (RNA), PNA and/or protein to provide a complex with a higher stability than when the carrier peptide does not have this criteria.

While not wishing to be confined to any specific theory, the inventors suggest this property of the carrier peptide component of the complex of the invention arises since the presence of the disulphide bond linking the two cysteine residues limits the flexibility of the carrier peptide and thus forms a more stable complex when in association with the second component (i.e, a ribonucleic acid (RNA), PNA and/or protein).

It can be appreciated to the skilled person that a wide variety of carrier peptide sequences can be used as component (i) of the complex of the invention.

There are several types of cell-penetrating peptides as reviewed in Bechara and Sagan (FEBS Lett. 2013 587:1693-1702). They are short peptides that have the capacity to cross cellular membranes without the need of recognition by specific receptors. In general, three types can be distinguished: natural occurring peptides, fusion of different natural occurring peptides and synthetic peptides.

Preferably, the cell-penetrating sequence is KKLFKKILKYL (SEQ ID NO: 11).

Further preferably the polycation sequence is HHCRGHTVHSHHHCIR (SEQ ID NO: 12). However as can be appreciated the position of the two cysteine residues within the polycation sequence can be changed to other locations within the polycation sequence.

A preferred embodiment of the invention is wherein the carrier peptide is that defined in SEQ ID 3.

The complex of the invention has much utility in delivering ribonucleic acid (RNA), PNA and/or protein to the plant cell.

In an embodiment of the complex of the invention, component (ii) comprises a protein, and the protein is a nuclease, a TALEN, peptide nucleic acid or a zinc finger transcription factor.

A nuclease is an enzyme capable of cleaving the phosphodiester bonds between monomers of nucleic acids. Nucleases variously effect single and double stranded breaks in their target molecules. There are two primary classifications based on the locus of activity. Exonucleases digest nucleic acids from the ends. Endonucleases act on regions in the middle of target molecules. They are further subcategorized as deoxyribonucleases and ribonucleases. The former acts on DNA, the latter on RNA.

TALEN is a protein secreted by Xanthomonas bacteria via their type Ill secretion system when they infect various plant species. These proteins can bind promoter sequences in the host plant and activate the expression of plant genes that aid bacterial infection. They recognize plant DNA sequences through a central repeat domain consisting of a variable number of 34 amino acid repeats. There is a one-to-one correspondence between the identity of two critical amino acids in each repeat and each DNA base in the target sequence.

This simple correspondence between amino acids in TALE and DNA bases in their target sites makes them useful for protein engineering applications, as it is possible to programme the TALE to recognize specific DNA sequences.

For example, Jankele and Svoboda (Briefings In Functional Genomics 13, 409-419) review the DNA binding specificity governed by the DNA binding domain and report that two polymorphic amino acid residues at positions 12 and 13 form the repeat-variable diresidue (RVD) in which the amino acid at position 13 is responsible for the preferential binding of the repeat module to a single specific nucleotide. Hence a protein can be programmed to bind to a specific DNA sequences by tandem array of the DNA binding domains.

Zinc finger transcription factor can be engineered to bind to a predetermined nucleotide sequence, for example via engineering (altering one or more amino acids) of the recognition helix region of a naturally occurring zinc finger protein.

The combination of cell-penetrating peptides with a monomeric guided nuclease allows the direct application of the nuclease as protein to the plant cell without the need to genetic transformation of first a DNA-molecule encoding the nuclease into the plant genome. Further, the application of a nuclease protein to regenerating plant cells allows the propagation of the genome modifications to the next generation without long tissue culture procedures or the need of additional generations.

Hence in preferred embodiments of the invention, the nuclease is a RNA guided nuclease, preferably Cas9 or Cpf1. More preferably the nuclease is Cas9.

As stated above, Cas9 is a component of the CRISPR/Cas system. CRISPR cutting properties can be used to disrupt genes in almost any organism's genome with unprecedented ease. Hence the complex of the invention allows the introduction the genome modifications into plant cells.

The complex of the invention may also comprise as component (ii) an RNA molecule.

CRISPR-Cas system relies on two main components: a guide RNA (gRNA) and CRISPR-associated (Cas) nuclease. The guide RNA is a specific RNA sequence that recognizes the target DNA region of interest and directs the Cas nuclease there for editing. The gRNA is made up of two parts: crispr RNA (crRNA), a 17-20 nucleotide sequence complementary to the target DNA, and a tracr RNA, which serves as a binding scaffold for the Cas nuclease. The CRISPR-associated protein is a non-specific endonuclease. It is directed to the specific DNA locus by a gRNA, where it makes a double-strand break. There are several versions of Cas nucleases isolated from different bacteria. The most commonly used one is the Cas9 nuclease from Streptococcus pyogenes. The crRNA part of the gRNA is the customizable component that enables specificity in every CRISPR experiment.

sgRNA is an abbreviation for “single guide RNA” As the name implies, an sgRNA is a single RNA molecule that contains both the custom-designed short crRNA sequence fused to the scaffold tracrRNA sequence. sgRNA can be synthetically generated or made in vitro or in vivo from a DNA template. While crRNAs and tracrRNAs exist as two separate RNA molecules in nature, sgRNAs have become the most popular format for CRISPR guide RNAs. Hence gRNA is the term that describes all CRISPR guide RNA formats, and sgRNA refers to the simpler alternative that combines both the crRNA and tracrRNA elements into a single RNA molecule.

Preferably the RNA molecule is a guide RNA or sgRNA molecule.

More preferably an embodiment of the complex of the invention is wherein component (ii) comprises Cas9 and a guide RNA.

It can be appreciated by the skilled person that the complex of the invention comprises two components. As shown herein in the accompanying examples the inventors made a series of complexes in which the ratios between the components was varied. Accordingly, a further embodiment of the invention is wherein the molar ratio of the carrier peptide to component (ii) is between 1:1 and 100:1. Preferably the molar ratio of the carrier peptide to component (ii) is 1:1, 5:1, 10:1, 20:1, 50:1 or 100:1.

A further aspect of the invention is a method of preparing a complex of the first aspect of the invention, comprising
(i) preparing a sample of the carrier peptide component;
(ii) preparing a sample of the ribonucleic acid (RNA), PNA and/or protein component;
(iii) mixing samples (i) and (ii) at room temperature;
(iv) allowing the resulting solution to incubate for 30 mins to 60 mins in the dark;
   wherein the molar ratio of the carrier peptide to component (ii) is between 1:1 and 100:1.

A further aspect of the invention is a method of introducing ribonucleic acid, PNA and/or protein to a target plant cell(s), comprising the step of bringing the complex of the first aspect of the invention into contact with the target plant cell(s).

Preferably the target plant cell is selected from the group comprising tobacco, carrot, maize, canola, rapeseed, cotton, palm, peanut, soybean, sunflower, wheat, Oryza sp., Arabidopsis sp., Ricinus sp., and sugarcane, cells.

Preferably the plant cell is from a tissue selected from the group consisting of embryo, meristematic, callus, explant, seedlings, pollen, leaves, anthers, roots, root tips, flowers, seeds, pods and stems.

The method of the invention can be used to deliver the complex of the invention into a target plant cell, where the constituents of component (ii) of the complex can act.

In a preferred embodiment of the invention, the plant cell is rice callus tissue, and wherein the complex of any of claims 1 to 8 is brought into contact with the callus tissue by incubating the callus tissue with the complex at −0.08 MPa for 1 min, then incubating the callus tissue with the complex at +0.08 MPa for 1 min, then incubating the callus tissue at 30° C. in the dark.

In a further preferred embodiment of the invention, the plant cell is soybean explant tissue, and wherein the complex of any of claims 1 to 8 is brought into contact with the soybean explant tissue by vacuum infiltration. Preferably the infiltration is performed for 15 minutes.

A further method of the invention provides a method effecting a genetic alteration in the genome of a plant cell comprising: (i) exposing the plant, or a tissue, cell or callus of a plant, to the complex of the first aspect of the invention,
wherein component (ii) of the complex comprises (a) an RNA-guided nuclease, and (b) at least one guide RNA or polynucleotide encoding a guide RNA;
wherein the at least one guide RNA is capable of directing the RNA-guided nuclease to a defined location in the genome, thereby effecting a genetic alteration at the defined location in the genome
wherein the genetic alteration is at least one alteration selected from the group consisting of insertion of at least one nucleotide, deletion of at least one nucleotide, or replacement of at least one nucleotide at the defined location in the genome or any combination thereof.

In a preferred embodiment the RNA-guided nuclease is Cas9.

In a further preferred embodiment the ratio of (a) the RNA-guided nuclease, and (b) at least one guide RNA is 0.5.

In a further preferred embodiment the molar charge of the carrier peptide to component (ii) is 30:1.

A further aspect of the invention provides a method of introducing ribonucleic acid, PNA and/or protein to rice plant cell(s).comprising the step of bringing a complex into contact with the target plant cell(s), wherein the complex comprises a first component: (i) a carrier peptide comprising a cell-penetrating sequence and a polycation sequence; and a second component (ii) a ribonucleic acid (RNA), PNA and/or protein, wherein the carrier peptide has the sequence defined in SEQ IS NO: 2.

Preferably the rice plant is rice callus tissue, and wherein the complex is brought into contact with the callus tissue by incubating the callus tissue with the complex at −0.08 MPa for 1 min, then incubating the callus tissue with the complex at +0.08 MPa for 1 min, then incubating the callus tissue at 30° C. in the dark.

### DEFINITIONS AND FURTHER DESCRIPTION OF THE INVENTION

It is to be understood that this invention is not limited to the particular methodology or protocols. It is also to be understood that the terminology used herein is for the purpose of describing particular embodiments only, and is not intended to limit the scope of the present invention which will be limited only by the appended claims. It must be noted that as used herein and in the appended claims, the singular forms “a,” “and,” and “the” include plural reference unless the context clearly dictates otherwise. Thus, for example, reference to “a vector” is a reference to one or more vectors and includes equivalents thereof known to those skilled in the art, and so forth. The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). As used herein, the word “or” means any one member of a particular list and also includes any combination of members of that list. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. For clarity, certain terms used in the specification are defined and used as follows:

The terms “domain”, “signature” and “motif’ are defined in the “definitions” section herein. Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp53-61, AAAI Press, Menlo Park; Hula et al., Nucl. Acids. Res. 32: D134-D137, (2004)), or Pfam (Bateman et al., Nucleic Acids Research 30 (1): 276-280 (2002)). A set of tools for in silica analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788 (2003)). Domains or motifs may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48:443-453) to find the global (i.e. spanning the complete sequences) alignment of two sequences that maximizes the number of matches and minimizes the number of gaps. The BLAST algorithm (Altschul et al. (1990) J Mol Biol 215:403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGA T: an application that generates similarity/identity matrices using protein or DNA sequences.). Minor manual editing may be performed to optimize alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters. For local alignments, the Smith-Waterman algorithm is particularly useful (Smith T F, Waterman M S (1981) J. Mol. Biol 147 (1); 195-7).

Performance of the methods of the invention results in plants having enhanced yield-related traits. In particular performance of the methods of the invention results in plants having increased yield, especially increased seed yield relative to control plants. The terms “yield” and “seed yield” are described in more detail herein. Reference herein to enhanced yield-related traits is taken to mean an increase in biomass (weight) of one or more parts of a plant, which may include aboveground (harvestable) parts and/or (harvestable) parts below ground. In particular, such harvestable parts are seeds, and performance of the methods of the invention results in plants having increased seed yield relative to the seed yield of control plants.

Taking corn as an example, a yield increase may be manifested as one or more of the following: increase in the number of plants established per square meter, an increase in the number of ears per plant, an increase in the number of rows, number of kernels per row, kernel weight, thousand kernel weight, ear length/diameter, increase in the seed filling rate (which is the number of filled seeds divided by the total number of seeds and multiplied by 100), among others. Taking rice as an example, a yield increase may manifest itself as an increase in one or more of the following: number of plants per square meter, number of panicles per plant, number of spikelets per panicle, number of flowers (florets) per panicle (which is expressed as a ratio of the number of filled seeds over the number of primary panicles), increase in the seed filling rate (which is the number of filled seeds divided by the total number of seeds and multiplied by 100), increase in thousand kernel weight, among others. The present invention provides a method for increasing yield.

In particular, the methods of the present invention may be performed under non-stress conditions or under conditions of mild drought to give plants having increased yield relative to control plants. As reported in Wang et al. (Planta (2003) 218:1-14), abiotic stress leads to a series of morphological, physiological, biochemical and molecular changes that adversely affect plant growth and productivity. Drought, salinity, extreme temperatures and oxidative stress are known to be interconnected and may induce growth and cellular damage through similar mechanisms. Rabbani et al. (Plant Physiol (2003) 133:1755-1767) describes a particularly high degree of “cross talk” between drought stress and high-salinity stress. For example, drought and/or salinisation are manifested primarily as osmotic stress, resulting in the disruption of homeostasis and ion distribution in the cell. Oxidative stress, which frequently accompanies high or low temperature, salinity or drought stress, may cause denaturing of functional and structural proteins. As a consequence, these diverse environmental stresses often activate similar cell signalling pathways and cellular responses, such as the production of stress proteins, up-regulation of anti-oxidants, accumulation of compatible solutes and growth arrest. The term “non-stress” conditions as used herein are those environmental conditions that allow optimal growth of plants. Persons skilled in the art are aware of normal soil conditions and climatic conditions for a given location. Plants with optimal growth conditions, (grown under non-stress conditions) typically yield in increasing order of preference at least 97%, 95%, 92%, 90%, 87%, 85%, 83%, 80%, 77% or 75% of the average production of such plant in a given environment. Average production may be calculated on harvest and/or season basis. Persons skilled in the art are aware of average yield productions of a crop.

The present invention clearly extends to any plant cell or plant produced by any of the methods described herein, and to all plant parts and propagules thereof. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced by the parent in the methods according to the invention.

The methods of the invention are advantageously applicable to any plant. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to a preferred embodiment of the present invention, the plant is a crop plant. Examples of crop plants include soybean, sunflower, canola, alfalfa, rapeseed, linseed, cotton, tomato, potato and tobacco. Further preferably, the plant is a monocotyledonous plant. Examples of monocotyledonous plants include sugarcane. More preferably the plant is a cereal. Examples of cereals include rice, maize, wheat, barley, millet, rye, triticale, sorghum, emmer, spelt, secale, einkorn, teff, milo and oats.

The production and use of plant gene-derived probes for use in genetic mapping is described in Bernatzky and Tanksley (1986) Plant Mol. Biol. Reporter 4:37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

Allelic variant: Alleles or allelic variants are alternative forms of a given gene, located at the same chromosomal position. Allelic variants encompass Single Nucleotide Polymorphisms (SNPs), as well as Small Insertion/Deletion Polymorphisms (INDELs). The size of INDELs is usually less than 100 bp. SNPs and IND Els form the largest set of sequence variants in naturally occurring polymorphic strains of most organisms.

Donor N A: the term “donor NA” or “doNA” means a nucleic acid comprising two homology arms each comprising at least 15 bases complementary to two different areas of at least 15 consecutive bases of the target NA, wherein said two homology arms are directly adjacent to each other or are separated by one or more additional bases.

The two different areas of the target NA to which the homology arms are complementary may be directly adjacent to each other or may be separated by additional bases of up to 20 kb, preferably up to 10 kb, preferably up to 5 kb, more preferably up to 3 kb, more preferably up to 2,5 kb, more preferably up to 2 kb.

In the event a homology arm comprises more than 15 bases, it may be 100% complementary to the target NA or it may be at least 75% complementary, preferably at least 80% complementary, more preferably at least 85% complementary, more preferably at least 90% complementary, more preferably at least 95% complementary, more preferably at least 98% complementary to the target NA, wherein the homology arm comprises at least one stretch of at least 15 bases that are 100% complementary to a stretch of the same number of consecutive bases in the target NA, preferably the homology arm comprises at least one stretch of at least 18 bases that are 100% complementary to a stretch of the same number of consecutive bases in the target NA, more preferably the homology arm comprises at least one stretch of at least 20 bases that are 100% complementary to a stretch of the same number of consecutive bases in the target NA, even more preferably the homology arm comprises at least one stretch of at least 25 bases that are 100% complementary to a stretch of the same number of consecutive bases in the target NA, even more preferably the homology arm comprises at least one stretch of at least 50 bases that are 100% complementary to a stretch of the same number of consecutive bases in the target NA.

The homology arms may have the same length and/or the same degree of complementarity to the target NA or may have different length and/or different degrees of complementarity to the target NA.

The homology arms may be directly adjacent to each other or may be separated by a nucleic acid molecule comprising at least one base not present between the regions in the target nucleic acid complementary to the homology arms.

Spacer N A: the term “spacer nucleic acid” or “spacer NA” means a nucleic acid comprising at least 12 bases 100% complementary to the target NA.

In the event the spacer NA comprises more than 12 bases, it may be at least 75% complementary to the target NA, preferably at least 80% complementary, more preferably at least 85% complementary, more preferably at least 90% complementary, more preferably at least 95% complementary, more preferably at least 98% complementary most preferably it is 100% complementary to the target NA, wherein the spacer NA comprises at least one stretch of at least 12 bases that are 100% complementary to a stretch of the same number of consecutive bases in the target NA, preferably the spacer NA comprises at least one stretch of at least 15 bases that are 100% complementary to a stretch of the same number of consecutive bases in the target NA, preferably the spacer NA comprises at least one stretch of at least 18 bases that are 100% complementary to a stretch of the same number of consecutive bases in the target NA, more preferably the spacer NA comprises at least one stretch of at least 20 bases that are 100% complementary to a stretch of the same number of consecutive bases in the target NA, even more preferably the spacer NA comprises at least one stretch of at least 25 bases that are 100% complementary to a stretch of the same number of consecutive bases in the target NA, even more preferably the spacer NA comprises at least one stretch of at least 50 bases that are 100% complementary to a stretch of the same number of consecutive bases in the target NA.

The spacer NA is covalently linked to a scaffold NA. If the scaffold NA is consisting of two nucleic acid molecules, the spacer is covalently linked to one molecule of a scaffold NA.

Scaffold N A: the scaffold nucleic acid or scaffold NA comprises a nucleic acid forming a secondary structure comprising at least one hairpin, preferably at least two hairpins and/or a sequence that is/are bound by the site directed nucleic acid modifying polypeptide. Such site directed nucleic acid modifying polypeptides are known in the art, for example in WO/2014/150624; WO/2014/204728. The scaffold NA further comprises two regions each comprising at least eight bases being complementary to each other, hence capable to hybridize forming a double-stranded structure. If said regions of at least eight bases complementary to each other are comprising more than eight bases, each region comprises at least eight bases that are complementary to at least eight bases of the other region.

The two complementary regions of the scaffold NA may be covalently linked to each other via a linker molecule forming a hairpin structure or may consist of two independent nucleic acid molecules.

Guide N A: the guide nucleic acid or guide NA or gNA comprises a spacer nucleic acid and a scaffold nucleic acid wherein the spacer NA and the scaffold NA are covalently linked to each other. In the event the scaffold NA consists of two molecules, the spacer NA is covalently linked to one molecule of the scaffold NA whereas the other molecule of the scaffold NA molecule hybridizes to the first scaffold NA molecule. Hence, a guide NA molecule may consist of one nucleic acid molecule or may consist of two nucleic acid molecules. Preferably the guide NA consists of one molecule.

Fusion NA: the fusion nucleic acid comprises donor NA and guide NA, wherein the guide NA and the donor NA are covalently linked to each other.

Site directed nucleic acid modifying polypeptide: By “site directed nucleic acid modifying polypeptide” “nucleic acid-binding site directed nucleic acid modifying polypeptide” or “site directed polypeptide” it is meant a polypeptide that binds nucleic acids and is targeted to a specific nucleic acid sequence. A site-directed nucleic acid modifying polypeptide as described herein is targeted to a specific nucleic acid sequence in the target nucleic acid either by mechanism intrinsic to the polypeptide or, preferably by the nucleic acid molecule to which it is bound. The nucleic acid molecule bound by the polypeptide comprises a sequence that is complementary to a target sequence within the target nucleic acid, thus targeting the bound polypeptide to a specific location within the target nucleic acid (the target sequence).

Most site directed nucleic acid modifying polypeptides introduce dsDNA breaks, but they may be modified to have only nicking activity or the nuclease activity may be inactivated. The site directed nucleic acid modifying polypeptides may be bound to a further polypeptide having an activity such as fluorescence or nuclease activity such as the nuclease activity of the Fokl polypeptide or a homing endonuclease polypeptide such as I-Scel.

Coding region: As used herein the term “coding region” when used in reference to a structural gene refers to the nucleotide sequences which encode the amino acids found in the nascent polypeptide as a result of translation of a mRNA molecule. The coding region is bounded, in eukaryotes, on the 5′-side by the nucleotide triplet “ATG” which encodes the initiator methionine, prokaryotes also use the triplets “GTG” and “TTG” as start codon. On the 3′-side it is bounded by one of the three triplets which specify stop codons (i.e., TAA, TAG, TGA). In addition a gene may include sequences located on both the 5′- and 3′-end of the sequences which are present on the RNA transcript. These sequences are referred to as “flanking” sequences or regions (these flanking sequences are located 5′ or 3′ to the non-translated sequences present on the mRNA transcript). The 5′-flanking region may contain regulatory sequences such as promoters and enhancers which control or influence the transcription of the gene. The 3′-flanking region may contain sequences which direct the termination of transcription, post-transcriptional cleavage and polyadenylation.

Complementary: “Complementary” or “complementarity” refers to two nucleotide sequences which comprise antiparallel nucleotide sequences capable of pairing with one another (by the base-pairing rules) upon formation of hydrogen bonds between the complementary base residues in the antiparallel nucleotide sequences. For example, the sequence 5′-AGT-3′ is complementary to the sequence 5′-ACT-3′. Complementarity can be “partial” or “total.” “Partial” complementarity is where one or more nucleic acid bases are not matched according to the base pairing rules. “Total” or “complete” complementarity between nucleic acid molecules is where each and every nucleic acid base is matched with another base under the base pairing rules. The degree of complementarity between nucleic acid molecule strands has significant effects on the efficiency and strength of hybridization between nucleic acid molecule strands. A “complement” of a nucleic acid sequence as used herein refers to a nucleotide sequence whose nucleic acid molecules show total complementarity to the nucleic acid molecules of the nucleic acid sequence.

Control plant(s): The choice of suitable control plants is a routine part of an experimental setup and may include corresponding wild type plants or corresponding plants without the gene of interest. The control plant is typically of the same plant species or even of the same variety as the plant to be assessed. The control plant may also be a nullizygote of the plant to be 40 assessed. Nullizygotes are individuals missing the transgene by segregation. A “control plant” as used herein refers not only to whole plants, but also to plant parts, including seeds and seed parts.

Endogenous: An “endogenous” nucleotide sequence refers to a nucleotide sequence, which is present in the genome of a wild type microorganism.

Enhanced expression: “enhance” or “increase” the expression of a nucleic acid molecule in a microorganism are used equivalently herein and mean that the level of expression of a nucleic acid molecule in a microorganism is higher compared to a reference microorganism, for example a wild type. The terms “enhanced” or “increased” as used herein mean herein higher, preferably significantly higher expression of the nucleic acid molecule to be expressed. As used herein, an “enhancement” or “increase” of the level of an agent such as a protein, mRNA or RNA means that the level is increased relative to a substantially identical microorganism grown under substantially identical conditions. As used herein, “enhancement” or “increase” of the level of an agent, such as for example a preRNA, mRNA, rRNA, tRNA, expressed by the target gene and/or of the protein product encoded by it, means that the level is increased 50% or more, for example 100% or more, preferably 200% or more, more preferably 5 fold or more, even more preferably 10 fold or more, most preferably 20 fold or more for example 50 fold relative to a suitable reference microorganism. The enhancement or increase can be determined by methods with which the skilled worker is familiar. Thus, the enhancement or increase of the nucleic acid or protein quantity can be determined for example by an immunological detection of the protein. Moreover, techniques such as protein assay, fluorescence, Northern hybridization, densitometric measurement of nucleic acid concentration in a gel, nuclease protection assay, reverse transcription (quantitative RT-PCR), ELISA (enzyme-linked immunosorbent assay), Western blotting, radioimmunoassay (RIA) or other immunoassays and fluorescence-activated cell analysis (FACS) can be employed to measure a specific protein or RNA in a microorganism. Depending on the type of the induced protein product, its activity or the effect on the phenotype of the microorganism may also be determined. Methods for determining the protein quantity are known to the skilled worker. Examples, which may be mentioned, are: the micro-Biuret method (Goa J (1953) Scand J Clin Lab Invest 5:218-222), the Folin-Ciocalteau method (Lowry O H et al. (1951) J Biol Chem 193:265-275) or measuring the absorption of CBB G-250 (Bradford M M (1976) Analyt Biochem 72:248-254).

Expression: “Expression” refers to the biosynthesis of a gene product, preferably to the transcription and/or translation of a nucleotide sequence, for example an endogenous gene or a heterologous gene, in a cell. For example, in the case of a structural gene, expression involves transcription of the structural gene into mRNA and—optionally—the subsequent translation of mRNA into one or more polypeptides. In other cases, expression may refer only to the transcription of the DNA harboring an RNA molecule.

Foreign: The term “foreign” refers to any nucleic acid molecule (e.g., gene sequence) which is introduced into a cell by experimental manipulations and may include sequences found in that cell as long as the introduced sequence contains some modification (e.g., a point mutation, the presence of a selectable marker gene, etc.) and is therefore different relative to the naturally-occurring sequence.

Functional fragment: the term “functional fragment” refers to any nucleic acid and/or protein which comprises merely a part of the full length nucleic acid and/or full length polypeptide of the invention but still provides the same function, i.e. the function of an AAT enzyme catalyzing the reaction of acryloyl-CoA and butanol to n-BA and CoA. Preferably, the fragment comprises at least 50%, at least 60%, at least 70%, at least 80%, at least 90% at least 95%, at least 98%, at least 99% of the sequence from which it is derived. Preferably, the functional fragment comprises contiguous nucleic acids or amino acids of the nucleic acid and/or protein from which the functional fragment is derived. A functional fragment of a nucleic acid molecule encoding a protein means a fragment of the nucleic acid molecule encoding a functional fragment of the protein.

Functional linkage: The term “functional linkage” or “functionally linked” is equivalent to the term “operable linkage” or “operably linked” and is to be understood as meaning, for example, the sequential arrangement of a regulatory element (e.g. a promoter) with a nucleic acid sequence to be expressed and, if appropriate, further regulatory elements (such as e.g., a terminator) in such a way that each of the regulatory elements can fulfill its intended function to allow, modify, facilitate or otherwise influence expression of said nucleic acid sequence. As a synonym the wording “operable linkage” or “operably linked” may be used. The expression may result depending on the arrangement of the nucleic acid sequences in relation to sense or antisense RNA. To this end, direct linkage in the chemical sense is not necessarily required. Genetic control sequences such as, for example, enhancer sequences, can also exert their function on the target sequence from positions which are further away, or indeed from other DNA molecules. Preferred arrangements are those in which the nucleic acid sequence to be expressed recombinantly is positioned behind the sequence acting as promoter, so that the two sequences are linked covalently to each other. In a preferred embodiment, the nucleic acid sequence to be transcribed is located behind the promoter in such a way that the transcription start is identical with the desired beginning of the chimeric RNA of the invention. Functional linkage, and an expression construct, can be generated by means of customary recombination and cloning techniques as described (e.g., Sambrook J, Fritsch E F and Maniatis T (1989); Silhavy et al. (1984) Experiments with Gene Fusions, Cold Spring Harbor Laboratory, Cold Spring Harbor (NY); Ausubel et al. (1987) Current Protocols in Molecular Biology, Greene Publishing Assoc. and Wiley Interscience; Gelvin et al. (Eds) (1990) Plant Molecular Biology Manual; Kluwer Academic Publisher, Dordrecht, The Netherlands). However, further sequences, which, for example, act as a linker with specific cleavage sites for restriction enzymes, or as a signal peptide, may also be positioned between the two sequences. The insertion of sequences may also lead to the expression of fusion proteins. Preferably, the expression construct, consisting of a linkage of a regulatory region for example a promoter and nucleic acid sequence to be expressed, can exist in a vector-integrated form or can be inserted into the genome, for example by transformation.

Gene: The term “gene” refers to a region operably linked to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (downstream) the coding region (open reading frame, ORF). The term “structural gene” as used herein is intended to mean a DNA sequence that is transcribed into mRNA which is then translated into a sequence of amino acids characteristic of a specific polypeptide.

Genome and genomic DNA: The terms “genome” or “genomic DNA” is referring to the heritable genetic information of a host organism. Said genomic DNA comprises the DNA of the nucleoid but also the DNA of the self-replicating plasmid.

Heterologous: The term “heterologous” with respect to a nucleic acid molecule or DNA refers to a nucleic acid molecule which is operably linked to, or is manipulated to become operably linked to, a second nucleic acid molecule to which it is not operably linked in nature, or to which it is operably linked at a different location in nature. A heterologous expression construct comprising a nucleic acid molecule and one or more regulatory nucleic acid molecule (such as a promoter or a transcription termination signal) linked thereto for example is a constructs originating by experimental manipulations in which either a) said nucleic acid molecule, or b) said regulatory nucleic acid molecule or c) both (i.e. (a) and (b)) is not located in its natural (native) genetic environment or has been modified by experimental manipulations, an example of a modification being a substitution, addition, deletion, inversion or insertion of one or more nucleotide residues. Natural genetic environment refers to the natural genomic locus in the organism of origin, or to the presence in a genomic library. In the case of a genomic library, the natural genetic environment of the sequence of the nucleic acid molecule is preferably retained, at least in part. The environment flanks the nucleic acid sequence at least at one side and has a sequence of at least 50 bp, preferably at least 500 bp, especially preferably at least 1,000 bp, very especially preferably at least 5,000 bp, in length. A naturally occurring expression construct—for example the naturally occurring combination of a promoter with the corresponding gene—becomes a transgenic expression construct when it is modified by non-natural, synthetic “artificial” methods such as, for example, mutagenization. Such methods have been described (U.S. Pat. No. 5,565,350; WO 00/15815). For example a protein encoding nucleic acid molecule operably linked to a promoter, which is not the native promoter of this molecule, is considered to be heterologous with respect to the promoter. Preferably, heterologous DNA is not endogenous to or not naturally associated with the cell into which it is introduced, but has been obtained from another cell or has been synthesized. Heterologous DNA also includes an endogenous DNA sequence, which contains some modification, non-naturally occurring, multiple copies of an endogenous DNA sequence, or a DNA sequence which is not naturally associated with another DNA sequence physically linked thereto. Generally, although not necessarily, heterologous DNA encodes RNA or proteins that are not normally produced by the cell into which it is expressed.

Homologue(s): “Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived. A deletion refers to removal of one or more amino acids from a protein. An insertion refers to one or more amino acid residues being introduced into a predetermined site in a protein. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to residues. Examples of N- or C-terminal fusion proteins or 15 peptides include the binding domain or activation domain of a transcriptional activator as used in the yeast two-hybrid system, phage coat proteins, (histidine)-6-tag, glutathione Stransferase-tag, protein A, maltose-binding protein, dihydrofolate reductase, Tag·100 epitope, c-myc epitope, FLAG®-epitope, lacZ, CMP (calmodulin-binding peptide), HA epitope, protein C epitope and VSV epitope.

A substitution refers to replacement of amino acids of the protein with other amino acids having similar properties (such as similar hydrophobicity, hydrophilicity, antigenicity, propensity to form or break a-helical structures or ˜-sheet structures). Amino acid substitutions are typically of single residues, but may be clustered depending upon functional constraints placed upon the polypeptide; insertions will usually be of the order of about 1 to 10 amino acid residues. The amino acid substitutions are preferably conservative amino acid substitutions. Conservative substitution tables are well known in the art (see for example Creighton (1984) Proteins. W. H. Freeman and Company (Eds) and Table 1 below).

Amino acid substitutions, deletions and/or insertions may readily be made using peptide synthetic techniques well known in the art, such as solid phase peptide synthesis and the like, or by recombinant DNA manipulation. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, OH), QuickChange Site Directed mutagenesis (Stratagene, San Diego, CA), PCR-mediated site-directed mutagenesis or other site-directed mutagenesis protocols.

Homologous recombination: Homologous recombination allows introduction in a genome of a selected nucleic acid at a defined selected position. Homologous recombination is a standard technology used routinely in biological sciences for lower organisms such as yeast or the moss Physcomitrella. Methods for performing homologous recombination in plants have been described not only for model plants (Offringa et al. (1990) EMBO J 9 (10): 3077-84) but also for crop plants, for example rice (Terada et al. (2002) Nat Biotech 20 (10): 1030-4; lida and Terada (2004) Curr Opin Biotech 15 (2): 132-8), and approaches exist that are generally applicable regardless of the target organism (Miller et al, Nature Biotechnol. 25, 778-785, 2007).

Hybridization: The term “hybridisation” as defined herein is a process wherein substantially complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

The term “stringency” refers to the conditions under which a hybridisation takes place. The stringency of hybridisation is influenced by conditions such as temperature, salt concentration, ionic strength and hybridisation buffer composition. Generally, low stringency conditions are selected to be about 30° C. lower than the thermal melting point (Tm) for the specific sequence at a defined ionic strength and pH. Medium stringency conditions are when the temperature is 20° C. below Tm, and high stringency conditions are when the temperature is 10° C. below Tm. High stringency hybridisation conditions are typically used for isolating hybridising sequences that have high sequence similarity to the target nucleic acid sequence. However, nucleic acids may deviate in sequence and still encode a substantially identical polypeptide, due to the degeneracy of the genetic code. Therefore, medium stringency hybridisation conditions may sometimes be needed to identify such nucleic acid molecules.

The “Tm” is the temperature under defined ionic strength and pH, at which 50% of the target sequence hybridises to a perfectly matched probe. The Tm is dependent upon the solution conditions and the base composition and length of the probe. For example, longer sequences hybridise specifically at higher temperatures. The maximum rate of hybridisation is obtained from about 16° C. up to 32° C. below Tm. The presence of monovalent cations in the hybridisation solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tm decreases about 1° C. per % base mismatch. The Tm may be calculated using the following equations, depending on the types of hybrids: DNA-DNA hybrids (Meinkoth and Wahl, Anal. Biochem., 138:267-284, 1984):

$$Tm = 81.5 °C + 16.6 \times log [ Na + ] a + 0.41 \% [ G / Cb ] - 500 \times [ Lc ] - 1 - 0.61 \times \% formamide$$

DNA-RNA or RNA-RNA hybrids:

$$Tm = 79.8 + 18.5 ( log 10 [ Na + ] a ) + 0.58 ( \% G / Cb ) + 11.8 ( \% G / Cb ) 2 - 820 / Lc$$

oligo-DNA or oligo-RNAd hybrids:
For <20 nucleotides: Tm=2 (In)
For 20-35 nucleotides: Tm=22+1.46 (In)
a or for other monovalent cation, but only accurate in the 0.01-0.4 M range.
b only accurate for % GC in the 30% to 75% range.
c L=length of duplex in base pairs.
d Oligo, oligonucleotide; In, effective length of primer=2×(no. of G/C)+(no. of A/T).

Non-specific binding may be controlled using any one of a number of known techniques such as, for example, blocking the membrane with protein containing solutions, additions of heterologous RNA, DNA, and SDS to the hybridisation buffer, and treatment with Rnase. For non-related probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1×SSC or at 42° C. in 1×SSC and 50% formamide, followed by washing at 65° C. in 0.3×SSC. Examples of medium stringency hybridisation conditions for DNA hybrids longer than 50 nucleotides encompass hybridisation at 50° C. in 4×SSC or at 40° C. in 6×SSC and 50% formamide, followed by washing at 50° C. in 2×SSC. The length of the hybrid is the anticipated length for the hybridising nucleic acid. When nucleic acids of known sequence are hybridised, the hybrid length may be determined by aligning the sequences and identifying the conserved regions described herein. 1×SSC is 0.15M NaCl and 15 mM sodium citrate; the hybridisation solution and wash solutions may additionally include 5×Denhardt's reagent, 0.5-1.0% SDS, 100 μg/ml denatured, fragmented salmon sperm DNA, 0.5% sodium pyrophosphate. Another example of high stringency conditions is hybridisation at 65° C. in 0.1×SSC comprising 0.1 SDS and optionally 5×Denhardt's reagent, 100 μg/ml denatured, fragmented salmon sperm DNA, 0.5% sodium pyrophosphate, followed by the washing at 65° C. in 0.3×SSC.

For the purposes of defining the level of stringency, reference can be made to Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

“Identity”: “Identity” when used in respect to the comparison of two or more nucleic acid or amino acid molecules means that the sequences of said molecules share a certain degree of sequence similarity, the sequences being partially identical.

Enzyme variants may be defined by their sequence identity when compared to a parent enzyme. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

The following example is meant to illustrate two nucleotide sequences, but the same calculations apply to protein sequences:

Seq A: AAGATACTG length: 9 bases
Seq B: GATCTGA length: 7 bases

Hence, the shorter sequence is sequence B.

Producing a pairwise global alignment which is showing both sequences over their complete lengths results in

Seq A: AAGATACTG-
||| |||
Seq B: --GAT-CTGA

The “I” symbol in the alignment indicates identical residues (which means bases for DNA or amino acids for proteins). The number of identical residues is 6.

The “-” symbol in the alignment indicates gaps. The number of gaps introduced by alignment within the Seq B is 1. The number of gaps introduced by alignment at borders of Seq B is 2, and at borders of Seq A is 1.

The alignment length showing the aligned sequences over their complete length is 10.

Producing a pairwise alignment which is showing the shorter sequence over its complete length according to the invention consequently results in:

Seq A: GATACTG-
||| |||
Seq B: GAT-CTGA

Producing a pairwise alignment which is showing sequence A over its complete length according to the invention consequently results in:

Seq A: AAGATACTG
||| |||
Seq B: --GAT-CTG

Producing a pairwise alignment which is showing sequence B over its complete length according to the invention consequently results in:

Seq A: GATACTG-
||| |||
Seq B: GAT-CTGA

The alignment length showing the shorter sequence over its complete length is 8 (one gap is present which is factored in the alignment length of the shorter sequence).

Accordingly, the alignment length showing Seq A over its complete length would be 9 (meaning Seq A is the sequence of the invention).

Accordingly, the alignment length showing Seq B over its complete length would be 8 (meaning Seq B is the sequence of the invention).

After aligning two sequences, in a second step, an identity value is determined from the alignment produced. For purposes of this description, percent identity is calculated by %-identity=(identical residues/length of the alignment region which is showing the respective sequence of this invention over its complete length)*100. Thus, sequence identity in relation to comparison of two amino acid sequences according to this embodiment is calculated by dividing the number of identical residues by the length of the alignment region which is showing the respective sequence of this invention over its complete length. This value is multiplied with 100 to give “%-identity”. According to the example provided above, %-identity is: for Seq A being the sequence of the invention (6/9)*100=66.7%; for Seq B being the sequence of the invention (6/8)*100=75%.

Isolated: The term “isolated” as used herein means that a material has been removed by the hand of man and exists apart from its original, native environment and is therefore not a product of nature. An isolated material or molecule (such as a DNA molecule or enzyme) may exist in a purified form or may exist in a non-native environment such as, for example, in a transgenic host cell. For example, a naturally occurring nucleic acid molecule or polypeptide present in a living cell is not isolated, but the same nucleic acid molecule or polypeptide, separated from some or all of the coexisting materials in the natural system, is isolated. Such nucleic acid molecules can be part of a vector and/or such nucleic acid molecules or polypeptides could be part of a composition, and would be isolated in that such a vector or composition is not part of its original environment. Preferably, the term “isolated” when used in relation to a nucleic acid molecule, as in “an isolated nucleic acid sequence” refers to a nucleic acid sequence that is identified and separated from at least one contaminant nucleic acid molecule with which it is ordinarily associated in its natural source. Isolated nucleic acid molecule is nucleic acid molecule present in a form or setting that is different from that in which it is found in nature. In contrast, non-isolated nucleic acid molecules are nucleic acid molecules such as DNA and RNA, which are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs, which encode a multitude of proteins. However, an isolated nucleic acid sequence comprising for example SEQ ID NO: 1 includes, by way of example, such nucleic acid sequences in cells which ordinarily contain SEQ ID NO: 1 where the nucleic acid sequence is in a genomic or plasmid location different from that of natural cells, or is otherwise flanked by a different nucleic acid sequence than that found in nature. The isolated nucleic acid sequence may be present in single- or double-stranded form. When an isolated nucleic acid sequence is to be utilized to express a protein, the nucleic acid sequence will contain at a minimum at least a portion of the sense or coding strand (i.e., the nucleic acid sequence may be single-stranded). Alternatively, it may contain both the sense and anti-sense strands (i.e., the nucleic acid sequence may be double-stranded).

Modulation: The term “modulation” means in relation to expression or gene expression, a process in which the expression level is changed by said gene expression in comparison to the control plant, the expression level may be increased or decreased. The original, unmodulated expression may be of any kind of expression of a structural RNA (rRNA, tRNA) or mRNA with subsequent translation. The term “modulating the activity” shall mean any change of the expression of the inventive nucleic acid sequences or encoded proteins, which leads to increased yield and/or increased growth of the plants.

Motif/Consensus sequence/Signature: The term “motif’ or “consensus sequence” or “signature” refers to a short conserved region in the sequence of evolutionarily related proteins. Motifs are frequently highly conserved parts of domains, but may also include only part of the domain, or be located outside of conserved domain (if all of the amino acids of the motif fall outside of a defined domain).

Non-coding: The term “non-coding” refers to sequences of nucleic acid molecules that do not encode part or all of an expressed protein. Non-coding sequences include but are not limited enhancers, promoter regions, 3′ untranslated regions, and 5′ untranslated regions.

Nucleic acids and nucleotides: The terms “nucleic acids” and “Nucleotides” refer to naturally occurring or synthetic or artificial nucleic acid or nucleotides. The terms “nucleic acids” and “nucleotides” comprise deoxyribonucleotides or ribonucleotides or any nucleotide analogue and polymers or hybrids thereof in either single- or double-stranded, sense or antisense form. Unless otherwise indicated, a particular nucleic acid sequence also implicitly encompasses conservatively modified variants thereof (e.g., degenerate codon substitutions) and complementary sequences, as well as the sequence explicitly indicated. The term “nucleic acid” is used inter-changeably herein with “gene”, “cDNA, “mRNA”, “oligonucleotide,” and “nucleic acid molecule”. Nucleotide analogues include nucleotides having modifications in the chemical structure of the base, sugar and/or phosphate, including, but not limited to, 5-position pyrimidine modifications, 8-position purine modifications, modifications at cytosine exocyclic amines, substitution of 5-bromo-uracil, and the like; and 2′-position sugar modifications, including but not limited to, sugar-modified ribonucleotides in which the 2′-OH is replaced by a group selected from H, OR, R, halo, SH, SR, NH2, NHR, NR2, or CN. Short hairpin RNAs (shRNAs) also can comprise non-natural elements such as non-natural bases, e.g., ionosin and xanthine, non-natural sugars, e.g., 2′-methoxy ribose, or non-natural phosphodiester linkages, e.g., methylphosphonates, phosphorothioates and peptides.

Nucleic acid sequence: The phrase “nucleic acid sequence” refers to a single- or double-stranded polymer of deoxyribonucleotide or ribonucleotide bases read from the 5′- to the 3′-end. It includes chromosomal DNA, self-replicating plasmids, infectious polymers of DNA or RNA and DNA or RNA that performs a primarily structural role. “Nucleic acid sequence” also refers to a consecutive list of abbreviations, letters, characters or words, which represent nucleotides. In one embodiment, a nucleic acid can be a “probe” which is a relatively short nucleic acid, usually less than 100 nucleotides in length. Often a nucleic acid probe is from about 50 nucleotides in length to about 10 nucleotides in length. A “target region” of a nucleic acid is a portion of a nucleic acid that is identified to be of interest. A “coding region” of a nucleic acid is the portion of the nucleic acid, which is transcribed and translated in a sequence-specific manner to produce into a particular polypeptide or protein when placed under the control of appropriate regulatory sequences. The coding region is said to encode such a polypeptide or protein.

Oligonucleotide: The term “oligonucleotide” refers to an oligomer or polymer of ribonucleic acid (RNA) or deoxyribonucleic acid (DNA) or mimetics thereof, as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases. An oligonucleotide preferably includes two or more nucleomonomers covalently coupled to each other by linkages (e.g., phosphodiesters) or substitute linkages.

Orthologue(s)/Paralogue(s): Orthologues and paralogues encompass evolutionary concepts used to describe the ancestral relationships of genes. Paralogues are genes within the same species that have originated through duplication of an ancestral gene; orthologues are genes from different organisms that have originated through speciation, and are also derived from a common ancestral gene.

Overhang: An “overhang” is a relatively short single-stranded nucleotide sequence on the 5′- or 3′-hydroxyl end of a double-stranded oligonucleotide molecule (also referred to as an “extension,” “protruding end,” or “sticky end”).

Plant: The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

Plants that are particularly useful in the methods of the invention include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Garica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Marus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticale sp., Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

Polypeptide: The terms “polypeptide”, “peptide”, “oligopeptide”, “polypeptide”, “gene product”, “expression product” and “protein” are used interchangeably herein to refer to a polymer or oligomer of consecutive amino acid residues.

Promoter: The terms “promoter”, or “promoter sequence” are equivalents and as used herein, refer to a DNA sequence which when operably linked to a nucleotide sequence of interest is capable of controlling the transcription of the nucleotide sequence of interest into RNA. A promoter is located 5′ (i.e., upstream), proximal to the transcriptional start site of a nucleotide sequence of interest whose transcription into mRNA it controls, and provides a site for specific binding by RNA polymerase and other transcription factors for initiation of transcription. The promoter does not comprise coding regions or 5′ untranslated regions. The promoter may for example be heterologous or homologous to the respective cell. A nucleic acid molecule sequence is “heterologous to” an organism or a second nucleic acid molecule sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety). Suitable promoters can be derived from genes of the host cells where expression should occur or from pathogens for this host.

Purified: As used herein, the term “purified” refers to molecules, either nucleic or amino acid sequences that are removed from their natural environment, isolated or separated. “Substantially purified” molecules are at least 60% free, preferably at least 75% free, and more preferably at least 90% free from other components with which they are naturally associated. A purified nucleic acid sequence may be an isolated nucleic acid sequence.

Regulatory element/Control sequence/Promoter: The terms “regulatory element”, “control sequence” and “promoter” are all used interchangeably herein and are to be taken in a broad context to refer to regulatory nucleic acid sequences capable of effecting expression of the sequences to which they are ligated. The term “promoter” typically refers to a nucleic acid control sequence located upstream from the transcriptional start of a gene and which is involved in recognising and binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative that confers, activates or enhances expression of a nucleic acid molecule in a cell, tissue or organ. A “plant promoter” comprises regulatory elements, which mediate the expression of a coding sequence segment in plant cells. Accordingly, a plant promoter need not be of plant origin, but may originate from viruses or micro-organisms, for example from viruses which attack plant cells. The “plant promoter” can also originate from a plant cell, e.g. from the plant which is transformed with the nucleic acid sequence to be expressed in the inventive process and described herein. This also applies to other “plant” regulatory signals, such as “plant” terminators. The promoters upstream of the nucleotide sequences useful in the methods of the present invention can be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is further more possible that the activity of the promoters is increased by modification of their sequence, or that they are replaced completely by more active promoters, even promoters from heterologous organisms. For expression in plants, the nucleic acid molecule must, as described above, be linked operably to or comprise a suitable promoter which expresses the gene at the right point in time and with the required spatial expression pattern. For the identification of functionally equivalent promoters, the promoter strength and/or expression pattern of a candidate promoter may be analysed for example by operably linking the promoter to a reporter gene and assaying the expression level and pattern of the reporter gene in various tissues of the plant. Suitable well-known reporter genes include for example beta-glucuronidase or beta-galactosidase. The promoter activity is assayed by measuring the enzymatic activity of the beta-glucuronidase or beta-galactosidase. The promoter strength and/or expression pattern may then be compared to that of a reference promoter (such as the one used in the methods of the present invention). Alternatively, promoter strength may be assayed by quantifying mRNA levels or by comparing mRNA levels of the nucleic acid used in the methods of the present invention, with mRNA levels of housekeeping genes such as 18S rRNA, using methods known in the art, such as Northern blotting with densitometric analysis of autoradiograms, quantitative real-time PCR or RTPCR (Heid et al., 1996 Genome Methods 6:986-994). Generally by “weak promoter” is intended a promoter that drives expression of a coding sequence at a low level. By “low level” is intended at levels of about 1/10,000 transcripts to about 1/100,000 transcripts, to about 1/500,0000 transcripts per cell. Conversely, a “strong promoter” drives expression of a coding sequence at high level, or at about 1/10 transcripts to about 1/100 transcripts to about 1/1000 transcripts per cell. Generally, by “medium strength promoter” is intended a promoter that drives expression of a coding sequence at a lower level than a strong promoter, in particular at a level that is in all instances below that obtained when under the control of a 35S CaMV promoter.

Significant increase: An increase for example in enzymatic activity, gene expression, productivity or yield of a certain product, that is larger than the margin of error inherent in the measurement technique, preferably an increase by about 10% or 25% preferably by 50% or 75%, more preferably 2-fold or −5 fold or greater of the activity, expression, productivity or yield of the control enzyme or expression in the control cell, productivity or yield of the control cell, even more preferably an increase by about 10-fold or greater.

Seed yield: Increased seed yield may manifest itself as one or more of the following: a) an increase in seed biomass (total seed weight) which may be on an individual seed basis and/or per plant and/or per square meter; b) increased number of flowers per plant; c) increased number of (filled) seeds; d) increased seed filling rate (which is expressed as the ratio between the number of filled seeds divided by the total number of seeds); e) increased harvest index, which is expressed as a ratio of the yield of harvestable parts, such as seeds, divided by the total biomass; and f) increased thousand kernel weight (TKW), and g) increased number of primary panicles, which is extrapolated from the number of filled seeds counted and their total weight. An increased TKW may result from an increased seed size and/or seed weight, and may also result from an increase in embryo and/or endosperm size. An increase in seed yield may also be manifested as an increase in seed size and/or seed volume. Furthermore, an increase in seed yield may also manifest itself as an increase in seed area and/or seed length and/or seed width and/or seed perimeter. Increased seed yield may also result in modified architecture, or may occur because of modified architecture.

Significant decrease: A decrease for example in enzymatic activity, gene expression, productivity or yield of a certain product, that is larger than the margin of error inherent in the measurement technique, preferably a decrease by at least about 5% or 10%, preferably by at least about 20% or 25%, more preferably by at least about 50% or 75%, even more preferably by at least about 80% or 85%, most preferably by at least about 90%, 95%, 97%, 98% or 99%.

Substantially complementary: In its broadest sense, the term “substantially complementary”, when used herein with respect to a nucleotide sequence in relation to a reference or target nucleotide sequence, means a nucleotide sequence having a percentage of identity between the substantially complementary nucleotide sequence and the exact complementary sequence of said reference or target nucleotide sequence of at least 60%, more desirably at least 70%, more desirably at least 80% or 85%, preferably at least 90%, more preferably at least 93%, still more preferably at least 95% or 96%, yet still more preferably at least 97% or 98%, yet still more preferably at least 99% or most preferably 100% (the later being equivalent to the term “identical” in this context). Preferably identity is assessed over a length of at least 19 nucleotides, preferably at least 50 nucleotides, more preferably the entire length of the nucleic acid sequence to said reference sequence. A nucleotide sequence “substantially complementary” to a reference nucleotide sequence hybridizes to the reference nucleotide sequence under low stringency conditions, preferably medium stringency conditions, most preferably high stringency conditions (as defined above).

TILLING: The term “TILLING” is an abbreviation of “Targeted Induced Local Lesions In Genomes” and refers to a mutagenesis technology useful to generate and/or identify nucleic acids encoding proteins with modified expression and/or activity. TILLING also allows selection of plants carrying such mutant variants. These mutant variants may exhibit modified expression, either in strength or in location or in timing (if the mutations affect the promoter for example). These mutant variants may exhibit higher activity than that exhibited by the gene in its natural form. TILLING combines high-density mutagenesis with high-throughput screening methods. The steps typically followed in TILLING are: (a) EMS mutagenesis (Redei G P and Koncz C (1992) In Methods in Arabidopsis Research, Koncz C, Chua N H, Schell J, eds. Singapore, World Scientific Publishing Co, pp. 16-82; Feldmann et al., (1994) In Meyerowitz E M, Somerville C R, eds, Arabidopsis. Cold Spring Harbor Laboratory Press, Cold Spring Harbor, NY, pp 137-172; Lightner J and Caspar T (1998) In J Martinez-Zapater, J Salinas, eds, Methods on Molecular Biology, Vol. 82. Humana Press, Totowa, NJ, pp 91-20 104); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (McCallum et al., (2000) Nat Biotechnol 18:455-457; reviewed by Stemple (2004) Nat Rev Genet 5 (2): 145-50).

Transgene: The term “transgene” as used herein refers to any nucleic acid sequence, which is introduced into the genome of a cell by experimental manipulations. A transgene may be an “endogenous DNA sequence,” or a “heterologous DNA sequence” (i.e., “foreign DNA”). The term “endogenous DNA sequence” refers to a nucleotide sequence, which is naturally found in the cell into which it is introduced so long as it does not contain some modification (e.g., a point mutation, the presence of a selectable marker gene, etc.) relative to the naturally-occurring sequence.

Transgenic: The term transgenic when referring to an organism means transformed, preferably stably transformed, with at least one recombinant nucleic acid molecule.

Transformation: The term “introduction” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed.

Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Biol 8:363-373); electroporation of protoplasts (Shillito R. D. et al. (1985) Bio/Technol 3, 1099-1102); microinjection into plant material (Crossway A et al., (1986) Mol. Gen Genet 202:179-185); DNA or RNA-coated particle bombardment (Klein T M et al., (1987) Nature 327:70) infection with (non-integrative) viruses and the like. Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least on the flower primordia. The plant is subsequently grown on until the seeds of the treated plant are obtained (Clough and Bent, Plant J. (1998) 16, 735-7 43). Methods for Agrobacterium-mediated transformation of rice include well known methods for rice transformation, such as those described in any of the following: European patent application EP 1198985 A 1, Aldemita and Hodges (Planta 199:612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14 (6): 7 45-50, 1996) or Frame et al. (Plant Physiol 129 (1): 13-22, 2002), which disclosures are incorporated by reference herein as if fully set forth. Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Malec. Biol. 42 (1991) 205-225). The nucleic acids or the construct to be expressed is preferably cloned into a vector, which is suitable for transforming Agrobacterium tumefaciens, for example pBin19 (Bevan et al., Nucl. Acids Res. 12 (1984) 8711). Agrobacteria transformed by such a vector can then be used in known manner for the transformation of plants, such as plants used as a model, like Arabidopsis (Arabidopsis thaliana is within the scope of the present invention not considered as a crop plant), or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Hofgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

In addition to the transformation of somatic cells, which then have to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic [Feldman, KA and Marks M D (1987). Mol Gen Genet 208:274-289; Feldmann K (1992). In: C Koncz, N-H Chua and J Shell, eds, Methods in Arabidopsis Research. Word Scientific, Singapore, pp. 27 4-289]. Alternative methods are based on the repeated removal of the inflorescences and incubation of the excision site in the center of the rosette with transformed agrobacteria, whereby transformed seeds can likewise be obtained at a later point in time (Chang (1994). Plant J. 5:551-558; Katavic (1994). Mol Gen Genet, 245:363-370). However, an especially effective method is the vacuum infiltration method with its modifications such as the “floral dip” method. In the case of vacuum infiltration of Arabidopsis, intact plants under reduced pressure are treated with an agrobacterial suspension [Bechthold, N (1993). CR Acad Sci Paris Life Sci, 316:1194-1199], while in the case of the “floral dip” method the developing floral tissue is incubated briefly with a surfactant-treated agrobacterial suspension [Clough, S J and Bent A F (1998) The Plant J. 16, 735-7 43]. A certain proportion of transgenic seeds are harvested in both cases, and these seeds can be distinguished from non-transgenic seeds by growing under the above described selective conditions. In addition the stable transformation of plastids is of advantages because plastids are inherited maternally is most crops reducing or eliminating the risk of transgene flow through pollen. The transformation of the chloroplast genome is generally achieved by a process which has been schematically displayed in Klaus et al., 25 2004 [Nature Biotechnology 22 (2), 225-229]. Briefly the sequences to be transformed are cloned together with a selectable marker gene between flanking sequences homologous to the chloroplast genome. These homologous flanking sequences direct site specific integration into the plastome. Plastidal transformation has been described for many different plant species and an overview is given in Bock (2001) Transgenic plastids in basic research and plant biotechnology. J Mol Biol. 2001 Sep. 21; 312 (3): 425-38 or Maliga, P (2003) Progress towards commercialization of plastid transformation technology. Trends Biotechnol. 21, 20-28. Further biotechnological progress has recently been reported in form of marker free plastid transformants, which can be produced by a transient co-integrated maker gene (Klaus et al., 2004, Nature Biotechnology 22 (2), 225-229).

Vector: As used herein, the term “vector” refers to a nucleic acid molecule capable of transporting another nucleic acid molecule to which it has been linked. One type of vector is a genomic integrated vector, or “integrated vector”, which can become integrated into the genomic DNA of the host cell. Another type of vector is an episomal vector, i.e., a plasmid or a nucleic acid molecule capable of extra-chromosomal replication. Vectors capable of directing the expression of genes to which they are operatively linked are referred to herein as “expression vectors”. In the present specification, “plasmid” and “vector” are used interchangeably unless otherwise clear from the context.

Wild type: The term “wild type”, “natural” or “natural origin” means with respect to an organism that said organism is not changed, mutated, or otherwise manipulated by man. With respect to a polypeptide or nucleic acid sequence, that the polypeptide or nucleic acid sequence is naturally occurring or available in at least one naturally occurring organism which is not changed, mutated, or otherwise manipulated by man.

A wild type of a microorganism refers to a microorganism whose genome is present in a state as before the introduction of a genetic modification of a certain gene. The genetic modification may be e.g. a deletion of a gene or a part thereof or a point mutation or the introduction of a gene.

The terms “production” or “productivity” are art-recognized and include the concentration of the fermentation product (for example, dsRNA) formed within a given time and a given fermentation volume (e.g., kg product per hour per liter). The term “efficiency of production” includes the time required for a particular level of production to be achieved (for example, how long it takes for the cell to attain a particular rate of output of a fine chemical).

The term “yield” or “product/carbon yield” is art-recognized and includes the efficiency of the conversion of the carbon source into the product (i.e., fine chemical). This is generally written as, for example, kg product per kg carbon source. By increasing the yield or production of the compound, the quantity of recovered molecules or of useful recovered molecules of that compound in a given amount of culture over a given amount of time is increased.

The term “recombinant microorganism” includes microorganisms which have been genetically modified such that they exhibit an altered or different genotype and/or phenotype (e. g., when the genetic modification affects coding nucleic acid sequences of the microorganism) as compared to the wild type microorganism from which it was derived. A recombinant microorganism comprises at least one recombinant nucleic acid molecule.

The term “recombinant” with respect to nucleic acid molecules refers to nucleic acid molecules produced by man using recombinant nucleic acid techniques. The term comprises nucleic acid molecules which as such do not exist in nature or do not exist in the organism from which the nucleic acid molecule is derived, but are modified, changed, mutated or otherwise manipulated by man. Preferably, a “recombinant nucleic acid molecule” is a non-naturally occurring nucleic acid molecule that differs in sequence from a naturally occurring nucleic acid molecule by at least one nucleic acid. A “recombinant nucleic acid molecules” may also comprise a “recombinant construct” which comprises, preferably operably linked, a sequence of nucleic acid molecules not naturally occurring in that order. Preferred methods for producing said recombinant nucleic acid molecules may comprise cloning techniques, directed or non-directed mutagenesis, gene synthesis or recombination techniques.

An example of such a recombinant nucleic acid molecule is a plasmid into which a heterologous DNA-sequence has been inserted or a gene or promoter which has been mutated compared to the gene or promoter from which the recombinant nucleic acid molecule derived. The mutation may be introduced by means of directed mutagenesis technologies known in the art or by random mutagenesis technologies such as chemical, UV light or x-ray mutagenesis or directed evolution technologies.

The term “directed evolution” is used synonymously with the term “metabolic evolution” herein and involves applying a selection pressure that favors the growth of mutants with the traits of interest. The selection pressure can be based on different culture conditions, ATP and growth coupled selection and redox related selection. The selection pressure can be carried out with batch fermentation with serial transferring inoculation or continuous culture with the same pressure.

The term “expression” or “gene expression” means the transcription of a specific gene(s) or specific genetic vector construct. The term “expression” or “gene expression” in particular means the transcription of gene(s) or genetic vector construct into mRNA. The process includes transcription of DNA and may include processing of the resulting RNA-product. The term “expression” or “gene expression” may also include the translation of the mRNA and therewith the synthesis of the encoded protein, i.e. protein expression.

### FIGURES

FIG. 1: Schematic diagram of the fusion peptide-based protein delivery system. The CPP+binding sequence is ionically combined with the protein citrine.

FIG. 2: Size and zeta-potential values of CPP-FP-Citrine complexes prepared at different molar ratios.

FIG. 3: Regeneration test of rice callus cells Rice seeds were grown on N6D medium for 5 days or 21 days with continuous light (a, b). Mature embryo-derived rice callus (c) was cut to small pieces in different sizes. Callus was then placed on regeneration medium an dkept cultured for one week (e, f). The smallest callus capable for plant generation was marked with boxes.

FIG. 4: Comparison of citrine fluorescence intensity per cell. Single cell areas were randomly selected, and their fluorescence intensity were calculated by confocal laser scanning microscopy (CLSM). Results from citrine delivery by BP1 (a), BP2 (b) 5 days (c), control at 5 d and 21 d (d, e) are shown and quantified (f). The scale bar is 10 μm.

FIG. 5: Observation of intracellular distribution of BP1-citrine (a-c) and BP2-citrine (d-f) complexes by CLSM. Arrows point to citrine (α), the cell membrane (β), complex of citrine and plasma membrane (γ). The scale bar is 10 μm.

FIG. 6: Observation of intracellular citrine delivery by BP1 and BP2 and citrine. The light spots represent citrine position. 3D structures were analysed by Imaris software and are Z-stack pictures from the CLMS.

FIG. 7: Quantification of citrine delivery by CPP-FP into rice callus. Western Blot analysis of the citrine extracted from 5 d rice callus at 72 hours after post infiltration with (1) water (Milli-q), (2) citrine without vacuum treatment (Citrine without physical treatment), (3) citrine with vacuum treatment (Citrine with physical treatment), (4) BP2-citrine (BP100CH7/Citrine), (5) BP1-citrine (BP100 (KH)9/Citrine), (6) positive control (Citrine protein 0.4 ng). Top panel, Western Blot using a-anti-citrine antibody for detection, Lower Panel, area intensity of Western Blot (Top Level) and quantification of area intensity normalized to positive control (6).

FIG. 8: PDI of citrine, BP1 (BP100 (KH)9-citrine, BP3 (BP1002K8)-citrine and BP2 (BP100CH7)-citrine complexes.

FIG. 9: Time course analysis of citrine delivery by BP1 and BP2 with 5 d callus.

FIG. 10: Time course analysis of citrine delivery by BP1 and BP2 with 21 d callus.

FIG. 11: Confocal sections of citrine delivery by BP1, BP2 and citrine without additional peptides. The arrows point out spots representing citrine positions.

FIG. 12: Plasmid Seq ID NO: 1 coding the expression cassette for visual marker dsRed.

FIG. 13: Delivery of plasmid Seq ID NO:1 into rice callus using BP1 and subsequent expression of the visual marker dsRed.

FIG. 14: Characterization of the Cas9/RNA complex of the different molar ratios of Cas9 protein and guide RNA.

FIG. 15: Size and zeta-potential values of CPP-FP/Cas9-gRNA complexes prepared at different molar ratios. Data are presented as mean±SD from triplicate tests. BP1=BP100 (KH), BP2=BP100CH7.

FIG. 16: Confocal images of Cas9-gRNA delivery into rice callus. Nuclei were visualized with Hoechst 33342 in blue, Cas9GFP were in green. The samples were prepared after 3-hour post-infiltration. The white arrows indicating the co-location of Cas9GFP and nuclei. The scale bar is 10 μm.

FIG. 17: Agarose gel analysis of T7 endonuclease assay on rice callus cells treated with the BP-Cas9 complex. The percentage is the ratio of mutant DNA mixed with untreated rice genomic DNA (wild type). The cleaved bands reveal the indels.

FIG. 18: Phenotypic analysis of rice plants regenerated from rice callus treated with CPP-FP/Cas9-gRNA complex. 1, BP2 with Target RNA3; 2, BP2 with Target RNA5; 3 BP1 with Target RNA5.

FIG. 19: Schematic representation of the glutathione-reducible peptide (BPCH7) and the proposed mechanism for intracellular delivery and subsequent pDNA release. BPCH7 (KKLFKKILKYLHHCRGHTVHSHHHCIR) can form sufficiently stable complex with plasmid DNA extracellularly and once delivered into the plant cell (endocytosis), the reductive intracellular environment, mediated mainly by GSH, induces cleavage of the intramolecular disulfide bond within the cyclic CH7 domain, thereby causing complex dissociation and subsequent release of pDNA in the cell for expression in the nucleus.

FIG. 20: Secondary structure contents of BPCH7, BPLH7, and BPKH in various solvents (reducing or non-reducing conditions). Analysis was performed using DichroWeb (CONTIN, dataset 4).

### EXAMPLES

### Chemicals and Common Methods

Unless indicated otherwise, cloning procedures carried out for the purposes of the present invention including restriction digest, agarose gel electrophoresis, purification of nucleic acids, ligation of nucleic acids, transformation, selection and cultivation of bacterial cells are performed as described (Sambrook J, Fritsch E F and Maniatis T (1989)). Sequence analyses of recombinant DNA are performed with a laser fluorescence DNA sequencer (Applied Biosystems, Foster City, CA, USA) using the Sanger technology (Sanger et al., 1977). Unless described otherwise, chemicals and reagents are obtained from Sigma Aldrich (Sigma Aldrich, St. Louis, USA), from Promega (Madison, WI, USA), Duchefa (Haarlem, The Netherlands) or Invitrogen (Carlsbad, CA, USA). Restriction endonucleases are from New England Biolabs (Ipswich, MA, USA) or Roche Diagnostics GmbH (Penzberg, Germany). Oligonucleotides are synthesized by Eurofins MWG Operon (Ebersberg, Germany).

### Identification of Sequences Related to the Nucleic Acid Sequence Used in the Methods of the Invention

Sequences (full length cDNA, ESTs or genomic) related to the nucleic acid sequence used in the methods of the present invention were identified amongst those maintained in the Entrez Nucleotides database at the National Center for Biotechnology Information (NCBI) and other databases using database sequence search tools, such as the Basic Local Alignment Tool (BLAST) (Altschul et al. (1990) J. Mol. Biol. 215:403-41 O; and Altschul et al. (1997) Nucleic Acids Res. 25:3389-3402). The program is used to find regions of local similarity between sequences by comparing nucleic acid or polypeptide sequences to sequence databases and by calculating the statistical significance of matches. For example, the polypeptide encoded by the nucleic acid used in the present invention was used for the TBLASTN algorithm, with default settings and the filter to ignore low complexity sequences set off. The output of the analysis was viewed by pairwise comparison, and ranked according to the probability score (E-value), where the score reflect the probability that a particular alignment occurs by chance (the lower the E-value, the more significant the hit). In addition to E-values, comparisons were also scored by percentage identity.

Percentage identity refers to the number of identical nucleotides (or amino acids) between the two compared nucleic acid (or polypeptide) sequences over a particular length. In some instances, the default parameters may be adjusted to modify the stringency of the search. For example the E-value may be increased to show less stringent matches. This way, short nearly exact matches may be identified.

### Yeast Strain, Media and Cultivation Conditions

The Saccharomyces cerevisiae strain used in the examples described is MaV203 (MATα, leu2-3,112, trp1-901, his3Δ200, ade2-101, gal4Δ, gal80Δ, SPAL10::URA3, GAL1::lacZ, HIS3UAS GAL1::HIS3@LYS2, can1R, cyh2R), commercialized by Life Technologies. Yeast was grown in Synthetic Minimal Media (SD Media) based upon Yeast Nitrogen Base supplemented with 2% glucose and lacking the appropriate auxotrophic compounds (ForMedium, United Kingdom). Cultures were grown at 30° C., either in a shaker or incubation oven.

Escherichia coli was used as propagation microorganism for all the plasmids used in our experiments, as well as for further propagation and maintenance of the modified targets. E. coli was grown according standard microbiological practices (Molecular Cloning: A Laboratory Manual, 3rd ed., Vols 1,2 and 3. J. F. Sambrook and D. W. Russell, ed., Cold Spring Harbor Laboratory Press, 2001). Plasmids containing the Cas9, guide RNA and donor NA included a pUC-based replication origin and ampicillin resistance gene for replication and maintenance in E. coli. Whereas GAL4 target plasmids contained a gentamicin resistance gene (Gmr).

### Rice Transformation

The Agrobacterium containing the expression vector was used to transform Oryza sativa plants. Mature dry seeds of the rice japonica cultivar Nipponbare were dehusked. Sterilization was carried out by incubating for one minute in 70% ethanol, followed by minutes in 0.2% HgCl2, followed by a 6 times 15 minutes wash with sterile distilled water. The sterile seeds were then germinated on a medium containing 2,4-D (callus induction medium). After incubation in the dark for four weeks, embryogenic, scutellum-derived calli were excised and propagated on the same medium. After two weeks, the calli were multiplied or propagated by subculture on the same medium for another 2 weeks. Embryogenic callus pieces were sub-cultured on fresh medium 3 days before co-cultivation (to boost cell division activity).

Agrobacterium strain LBA4404 containing the expression vector was used for co-cultivation. Agrobacterium was inoculated on AB medium with the appropriate antibiotics and cultured for 3 days at 28° C. The bacteria were then collected and suspended in liquid co-cultivation medium to a density (QD500) of about 1. The suspension was then transferred to a Petri dish and the calli immersed in the suspension for 15 minutes. The callus tissues were then blotted dry on a filter paper and transferred to solidified, co-cultivation medium and incubated for 3 days in the dark at 25° C. Co-cultivated calli were grown on 2,4-D-containing medium for 4 weeks in the dark at 28° C. in the presence of a selection agent. During this period, rapidly growing resistant callus islands developed. After transfer of this material to a regeneration medium and incubation in the light, the embryogenic potential was released and shoots developed in the next four to five weeks. Shoots were excised from the calli and incubated for 2 to 3 weeks on an auxin-containing medium from which they were transferred to soil. Hardened shoots were grown under high humidity and short days in a greenhouse.

Approximately 35 independent TO rice transformants were generated for one construct. The primary transformants were transferred from a tissue culture chamber to a greenhouse. After a quantitative PCR analysis to verify copy number of the T-DNA insert, only single copy transgenic plants that exhibit tolerance to the selection agent were kept for harvest of T1 seed. Seeds were then harvested three to five months after transplanting. The method yielded single locus transformants at a rate of over 50% (Aldemita and Hodges1996, Chan et al. 1993, Hiei et al. 1994).

### Soybean Transformation

Soybean is transformed according to a modification of the method described in the Texas A&M patent U.S. Pat. No. 5,164,310. Several commercial soybean varieties are amenable to transformation by this method. The cultivar Jack (available from the Illinois Seed foundation) is commonly used for transformation. Soybean seeds are sterilised for in vitro sowing. The hypocotyl, the radicle and one cotyledon are excised from seven-day old young seedlings. The epicotyl and the remaining cotyledon are further grown to develop axillary nodes. These axillary nodes are excised and incubated with Agrobacterium tumefaciens containing the expression vector. After the cocultivation treatment, the explants are washed and transferred to selection media. Regenerated shoots are excised and placed on a shoot elongation medium. Shoots no longer than 1 cm are placed on rooting medium until roots develop. The rooted shoots are transplanted to soil in the greenhouse. T1 seeds are produced from plants that exhibit tolerance to the selection agent and that contain a single copy of the T-DNA insert.

### Example 1 Generation of Cell Penetrating Peptides and Reporter Complex

Cell-penetrating peptides (CPPs), also called protein transduction domains, are short peptides that facilitate the transport of cargo molecules through membranes to gain access to the cells. In many cases, CPPs are coupled to cargo molecules through covalent conjugation, forming CPP-cargo complexes. To date, DNA, RNA, nanomaterials and proteins such as antibodies were reported as cargo molecules. Most studies of the complex of CPPs and protein have contributed to the applications in mammalian cells, whereas only very limited studies have focused on plant cells. This could be due to, unlike the nucleotides, nanomaterials and the antibodies, native proteins are large molecules with specific folding structure and surface charges different from one another. Additionally, the complicated cell wall structure of plant shows intransigence on internalization such big cargo molecules, and the slight negatively net charge of the cellulose, could reduce interaction between the CPP and lipid bilayer by the physical and the chemical manners. The plant cells are mainly contains cellulose, hemicellulose and pectin. These biochemical compositions are changing during the plant growth, indicating that we need to optimize various conditions to achieve delivery of native protein into plant cells.

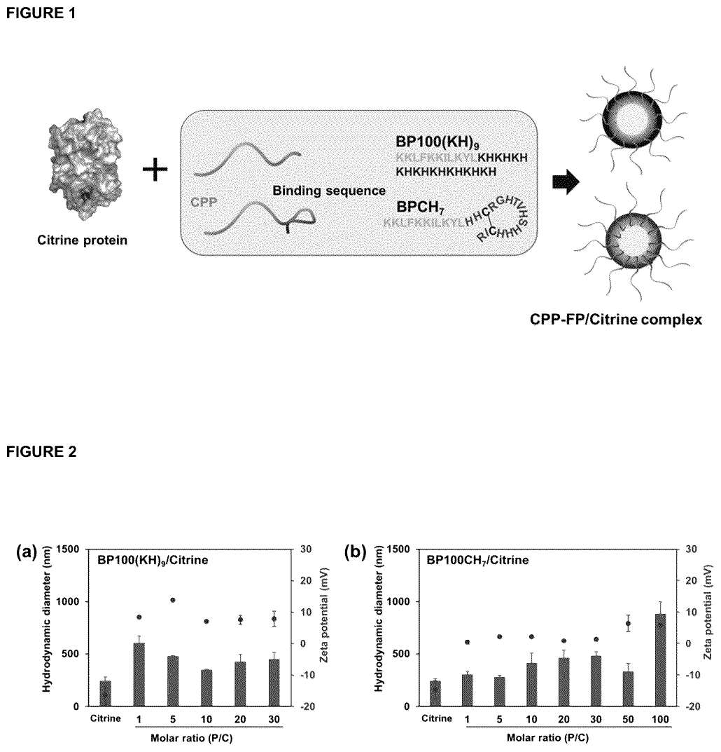

BP1 (BP100 (KH)₉ (KKLFKKILKYLKHKHKHKHKHKHKHKHKH) and BP2 (BP100CH₇ (KKLFKKILKYLHHCRGHTVHSHHHCIR) are fusion peptides containing CPP and cationic sequences (FIG. 1), which are designed as a stimulus-response peptides and could release the cargo molecules (peptides, protein, RNA, DNA) into the cytoplasm. Citrine was used as reporter molecule to detect successful delivery into plant cells.

The Citrine protein was prepared and purified use the same method as our previous work (Ng et al. Intracellular delivery of proteins via fusion peptides in intact plants. 2016; 1-19). To prepare the CPP-FP-Citrine complexes, 2 μg Citrine (1 mg/mL) was mixed with CPP-FP (1 mg/ml) at various molar ratios. For the BP100 (KH)₉-Citrine was prepared in the molar ratios at 1, 5, 10, 20 and 30, whereas the BP100CH₇-Citrine was prepared in the molar ratio at 1, 5, 10, 20, 30, 50, and 100. The complex solutions were pipetted gently and incubated at RT for 30 min in the dark. This solution was followed adjusted to final volume of 100 μL by adding autoclaved Milli-q water, and then continuing incubation under the same condition for another 30 min. After 10-fold dilution, each solution was repeat pipetted and characterized immediately. The Zetasizer Nano-ZS (Malvern Instruments, Ltd., Worcestershire, UK) was used for analysis the size, polydispersity index (PDI) and zeta potential as previously reported (Ngg et al. 2016).

### Example 2 Plant Growth Condition, Embryogenic Callus Induction and Recipient Cells

Mature dry seeds of Oryza sativa, cv. Nipponbare (O. sativa) were surface-sterilized with 70% ethanol (v/v) for 1 min, followed by 30 min in 50% (v/v) commercial bleach with rotation at 20 rpm. Seeds were then washed 8-10 times with sterile distilled water and dried on autoclaved Kimwipes (3 mm) for 5 min. For callus induction, sixteen seeds were inoculated petri plate on callus induction medium (N6D) and incubated at 30° C. with continuous light in a plant bio-incubator (TOMY CLE-303 cultivation chamber Tokyo, Japan). N6D was prepared using basal 30 g/L lactose, 0.3 g/L casamino acid, 2.8 g/L L-proline, 2 mg/L 2,4-dichlorophenoxyacetic acid (2,4-D), 4.0 g/L CHO (N6) basal salt mix, gelled with 4 g/L phytagel and pH adjusted to 5.8 before autoclaving. After 5-day cultivation, the callus was cut into approximately four equal parts. This callus was used as the 5-day callus in this work. On the other hand, after 21-day cultivation, the self-shedding callus was collected and used as the 21-day callus. For the callus regeneration test, the callus was cut to small pieces in different sizes, then transferred onto a regeneration medium and incubated at 30° C. with continuous light for 7 days. The callus which generate green plant was considered possess regeneration ability. The regeneration medium was prepared based on using 4 g/L MS powder with vitamin, supplied with 30 g/L sorbitol, 30 g/L sucrose, 4 g/L casamino acid, 2 mg/L 2,4-D and 2 mg/L 1-Naphthaleneacetic acid (NAA), gelled with 4 g/L phytagel and pH was adjusted to 5.8 before autoclaving. The chemicals used in this research are purchased from Sigma (Sigma-Aldrich, MO, USA) and Wako (Wako, Pure Chemical, Tokyo, Japan).

### Example 3 Penetration of CPP-FP-Citrine Complexes into Rice Callus Cell

10 mg rice callus (5-day or 21-day) was immersed in 100 μL of fresh prepared complex solutions in a screw cap tube. Subsequently depressurized the solution at −0.08 MPa for 1 min and compressed at +0.08 MPa for 1 min. After this treatment, the callus was transferred onto the N6D medium, and incubated at 30° C. in the dark until use.

### Example 4 Intracellular Uptake and Distribution Analysis of CPP-FP-Citrine Complexes

Confocal laser scanning microscopy (CLSM, ZeissLSM 700, Carl Zeiss, Oberkochen, Germany) was used to evaluate the intracellular uptake of CPP-FP-Citrine every 24 hours. Before the observation by CLSM, the callus on N6D was transferred into a 1.5 mL Eppendorf tube and washed thoroughly with Milli-q water contains 0.1% tween 20 for five times to remove the Citrine on cell surface. Thereafter, the callus was cut into tiny pieces and mounted on glass slides, then covered with coverslips. CPP-FP-Citrine was detected by setting the excitation at 488 nm and emission in a range of 505-600 nm. For distinguish plasma membrane region and cytoplasm region, the callus was additionally incubated with FM4-64 (20 UM, 20 min at RT) for cell membrane stain, and detected by setting the excitation at 405 nm and emission at 560-700. Furthermore, quantification of the intracellular Citrine by western blot immunoassay. After 72-hour post-infiltration, the protein was extracted from rice callus. The rice callus was frozen with liquid nitrogen, and then grinding in a mortar into powder. 50 μl of 10 mM Tris-HCL buffer (pH 7.4) containing 10 μL Halt protease inhibitor cocktail (Thermo Scientific, MA, USA) was added to 0.1 g callus powder, mixed well and incubated on ice for 1 hour. After centrifuge at 150 rpm, 4° C. for 20 min, the supernatant was collected and used as the cell extraction. 10 μL of the cell extraction was subjected to a 4-20% sodium dodecyl sulfate polyacrylamide electrophoresis gels (SDS-PAGE, Bio-Rad, California, USA). Proteins were then transferred onto a PVDF membranes (Bio-Rad, California, USA) using a semi-dry transfer cell (Bio-Rad, California, USA) at 10 V for 1 hour. The immunodetection of Citrine was performed with a rabbit polyclonal antibody (GFP antibody NB600-308, Novus Biologicals, Co, USA, dilution to 1:2000). IgG goat anti-rabbit antibody conjugated with horse radish peroxidase was used as a secondary antibody (goat anti-rabbit IgG H&L HRP ab6789, Abcam, Cambridge, UK, dilution to 1:20000). Luminescent image analyzer (LAS-3000, Fujifilm, Tokyo, Japan) was used to visualize the Citrine.

### Example 5 Characterization of CPP-FP-Citrine Complexes

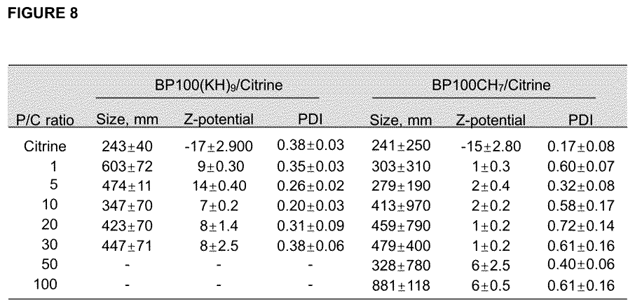

To discuss the effects of CPP-FP/Citrine (P/C) molar ratio in forming the complex, we firstly prepared the complexes of CPP-FP-Citrine in a range of 1 to 30. The results revealed that without the CPP-FP decoration, hydrodynamic diameter of Citrine was approximately 240 nm (FIG. 2a-b). By connection of the CPP-FPs, the hydrodynamic diameter and the PDI for BP100 (KH)9-Citrine were decreased at the P/C ratio from 1 to 10, and increased at the P/C ratio from 10 to 30. At a P/C ratio of 1, the complexes were showed large diameters and PDI values (FIG. 2a, FIG. 8), suggesting the small amount of the positively charged CPP-FP disturbed the electrostatic equilibrium of Citrine in solution, the heterogeneous surface charged CPP-FP-Citrine complexes are more accessible to aggregate. However, this phenomenon was countered by increasing the P/C ratio, the complex diameters and PDI were uniform and well distributed at a P/C ratio of 10 for BP100 (KH)9-Citrine (FIG. 2a, FIG. 8). Meanwhile, by additional of CPP-FP, the zeta potential of the complexes changed from negative to positive in all results of BP100 (KH)9-Citrine (FIG. 2a). On the other hand, The BP100CH7-Citrine did not show a significant difference in diameters and PDI, but a negative zeta potential (FIG. 2b, FIG. 8) at the P/C ratio from 1 to 30 (FIG. 2b). In order to prepare the positively charged CPP-FP-Citrine complex, we increased the P/C ratio to 100. Under this condition, we obtained the complex with diameter of 880 nm and the zeta potential of 5.8 (FIG. 2b). Based on these results, we suggest the P/C ratio should be optimum in size, PDI and surface charge for each event

### Example 6 Regeneration of the Rice Callus in Different Growth Stages

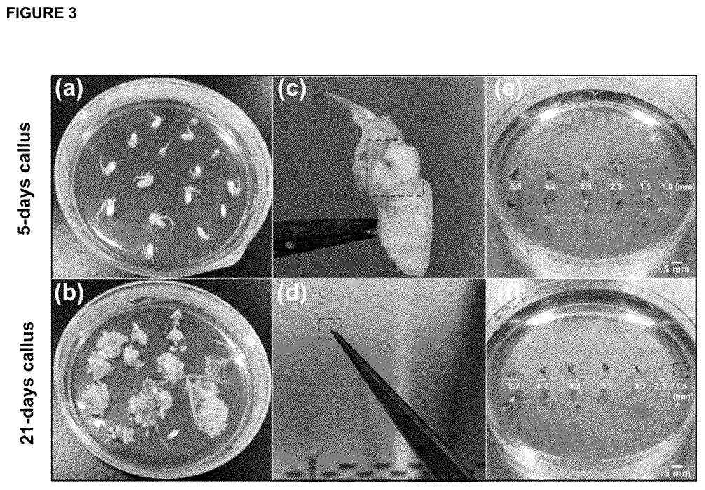

To assess whether the callus cell in different growth stages are distinctly sensitive to CPP-FP-Citrine delivery, we use 5-day and 21-day rice callus cells as the recipient cells (FIG. 3a-d). To enhance the superficial area of the callus for efficient connection with CPP-FPs, the callus was cut into small pieces. However, damage of cutting callus can affect the plant regeneration. We therefore tested the regeneration of the callus cells in different sizes. The smallest size of the callus that can generate plants was marked in the red box (FIG. 3e, f). The results suggested that the 5-day callus could generate plant from the callus size of 2 mm. Meanwhile, the 21-day callus possessed excellent regeneration ability, and could generated plant from the callus size smaller than 0.5 mm.

### Example 7 Selection of CPP-FP for Citrine Delivery with Rice Callus Cells

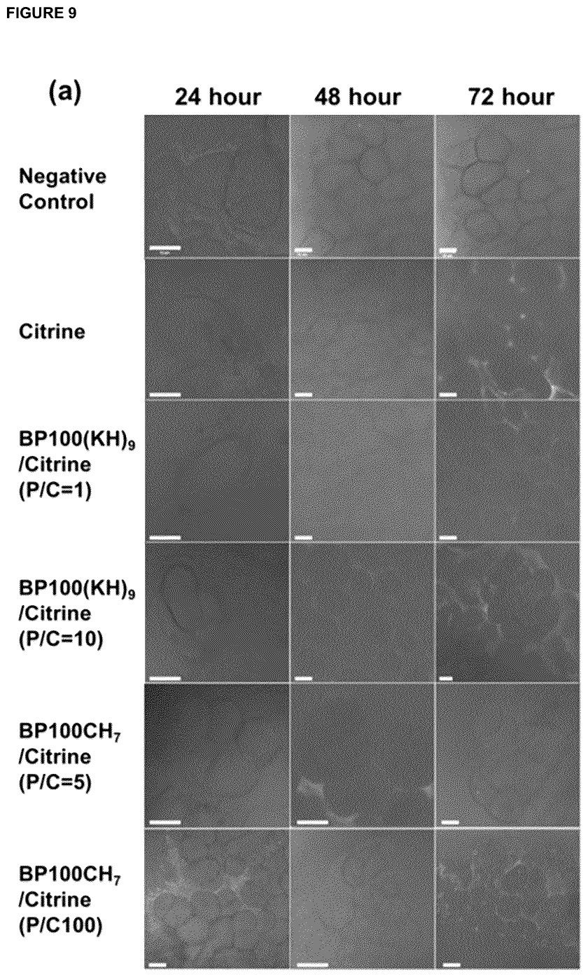

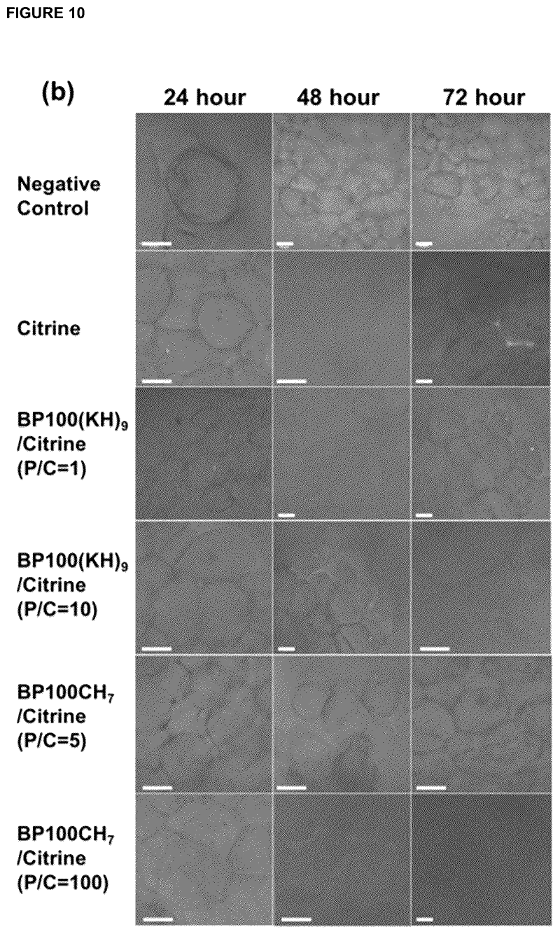

To clear the effect on CPP-FP for Citrine delivery in rice callus, two types of complexes for each CPP-FP were prepared. One is smaller diameter with low positive charges (P/C ratio of 1 for BP100 (KH), and 5 for BP100CH7), the other one is larger diameter with high positive charges (P/C ratio of 10 for BP100 (KH), and 100 for BP100CH7). The images were captured for observation of Citrine fluorescence every 24-hours by CLSM. For using the 5-day callus cells, the Citrine fluorescence inside of the cells was first observed at 48-hours after infiltration by the BP100 (KH)₉ at a P/C ratio of 10, this fluorescence was kept to 72-hours (FIG. 9). Likewise, the Citrine fluorescence inside of the cells was observed by BP100 (KH)₉ at P/C ratio of 1 and BP100CH7 at P/C ratio of 100 after 72-hours (FIG. 9). While those by using 21-day rice callus, the Citrine florescence was observed only in BP100 (KH), at a P/C ratio of 10 after 48-hours (FIG. 10). In addition, infiltration with Citrine only showed florescence in the cell joint part between cells at 72-hours (FIG. 9, FIG. 10), and the negative control which infiltration with Milli-q water showed no florescence in any test. These results indicated that BP100 (KH), and BP100CH₇ had the capacity to deliver Citrine in to rice callus.

### Example 8 Cell Penetration Efficiency

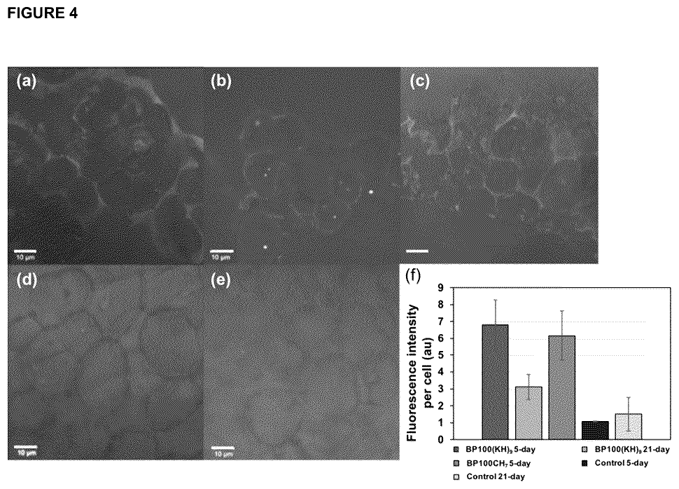

To evaluate the penetration efficiency with the recipient cells in growth stage, we compared the Citrine fluorescence intensity per cell area (FIG. 4a-e). The 5-day and 21-day callus were infiltrated with BP100 (KH)₉-Citrine at P/C of 10 and BP100CH₇-Citrine at P/C of 100. After 72-hours incubation on N6D medium, their fluorescence intensity was quantified by CLSM for each cell.

The average value from ten tests was calculated (FIG. 4f). The result revealed, for BP100 (KH)₉-Citrine delivery, the 5-day callus cell showed significantly higher fluorescence intensity, the value is as twice as it showed in 21-day callus (FIG. 4f). Moreover, the BP100 (KH)₉-Citrine and BP100CH₇-Citrine showed a similar fluorescence intensity value after post-infiltration in 5-day callus cell (FIG. 4f). This These results suggest, compare in contrast to the 21-day callus cell, the 5-day callus cell tissue was better compromised to internalize foreigner cargos via CPP-mediated transmission. Beside, and BP100 (KH), and BP100CH₇were have similar capability in to delivering Citrine into 5-day callus cell.

### Example 9 Citrine Distribution in Callus Cells

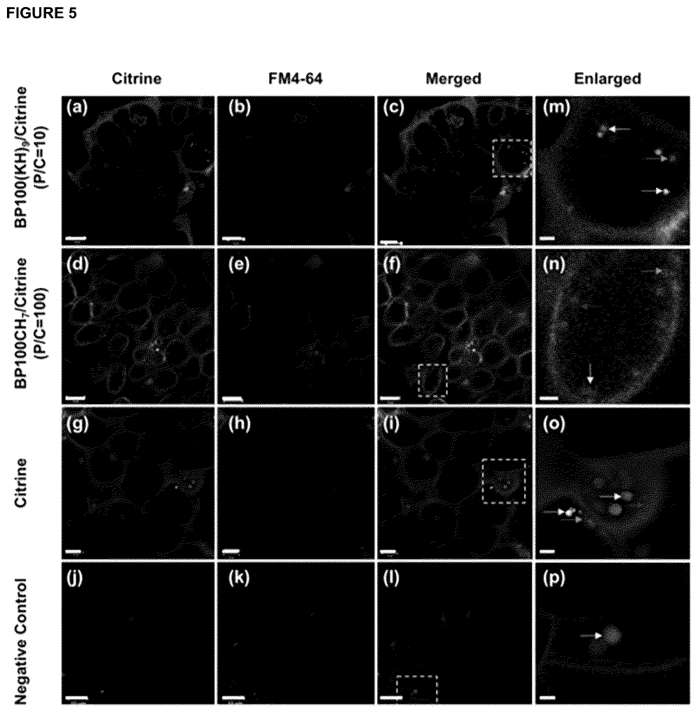

To confirm the distribution of Citrine in callus cell, BP100 (KH)₉-Citrine at P/C ratio of 10, BP100CH₇-Citrine at P/C ratio of 100, Citrine only and Milli-q water were infiltration into 5-day callus cell. After 72-hours incubation, the callus was stained with FM 4-64 (FIG. 5) for indication of the cell membrane. The merged CLSM imagines suggesting an overlapped fluorescence color (orange) from Citrine and FM 4-64 by BP100 (KH)₉-Citrine and BP100CH₇-Citrine (FIG. 5a-f), suggesting some Citrine was located in the cell membrane region. Further, the expanding figures (FIG. 5m, n) showed yellow and orange spots inside of the cells, indicating the Citrine and Citrine-cell membrane complex were located inside of the cell. Besides, some red spots also exhibited inside of the cell, that should be the cell membrane stained by MF4-64. Meanwhile, in the result of the Citrine only, it is also showed a small number of those spots inside of the cell (FIG. 5g-l, o). Additional, some fluorescence spots (FIG. 5m, white arrow), which differs from Citrine was observed. This fluorescence was considered from symbiotic bacteria in rice cell, since similar fluorescence was observed in negative control (FIG. 5j-l, p).

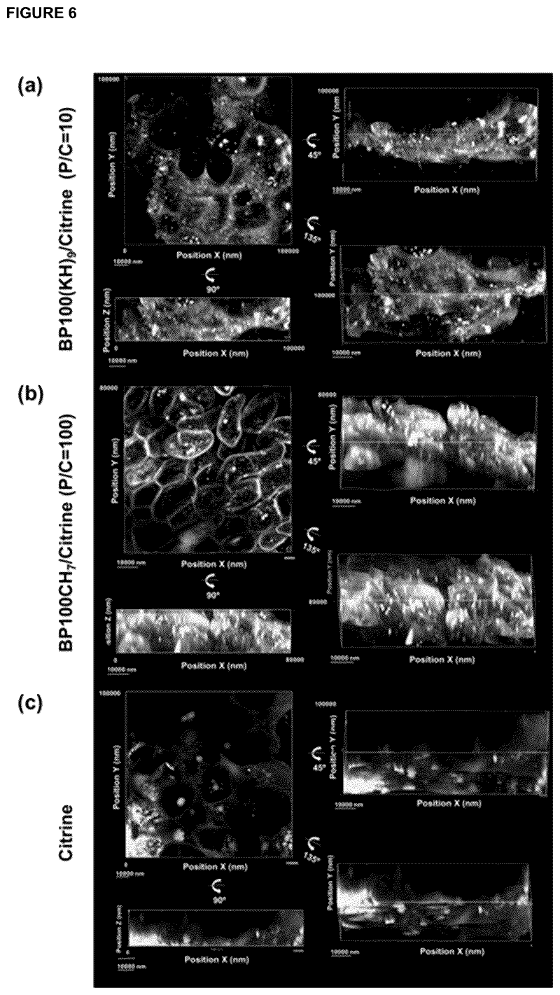

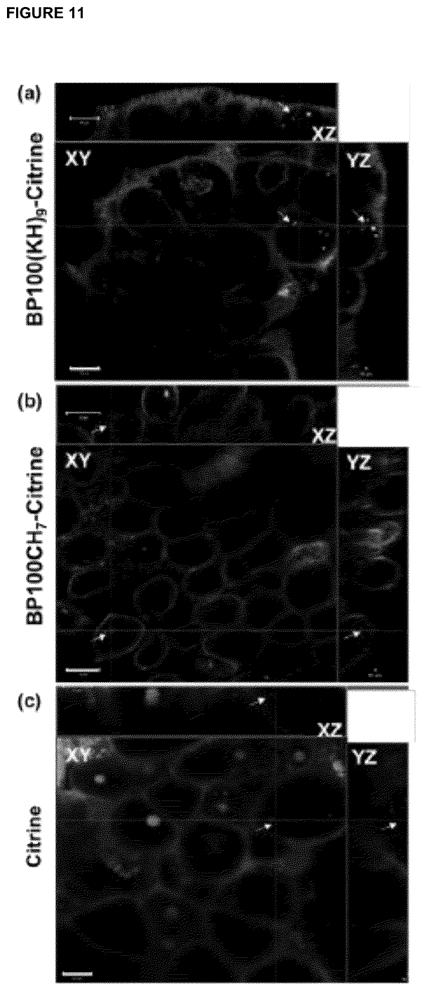

To further verify the Citrine delivered inside of the cells, we subjected these results from CLSM to Imaris to generate 3D images, then we could determine the distribution of Citrine in different angle of views (FIG. 6). The results of BP100 (KH)₉-Citrine and BP100CH₇-Citrine showed the yellow spots of Citrine were full of the cells. Further, most of these spots were under layers of the cell membrane (FIG. 6a-b). Moreover, consistent with FIG. 5, the spots in red and orange also observed inside of the cell. Indicating the Citrine were embroiled into the cell by endocytosis. While the result of delivery Citrine without CPP-FP also showed few spots inside of the cell (FIG. 6c), indicting without CPP-FP, less Citrine could be uptake into the cell by the endocytosis. Those results also supposed by a confocal optical sections analysis (FIG. 11). We randomly selected one yellow spot located in xy plane inside of the cells of BP100 (KH)₉-Citrine and BP100CH₇-Citrine. Clearly, the selected spot also displayed in xz and yz planes. These results indicating, delivery by BP100 (KH), and BP100CH₇, Citrine was located in cell membrane and the cytoplasm regions of the cells.

### Example 10 Quantification of Citrine Delivery by BP1 and BP2

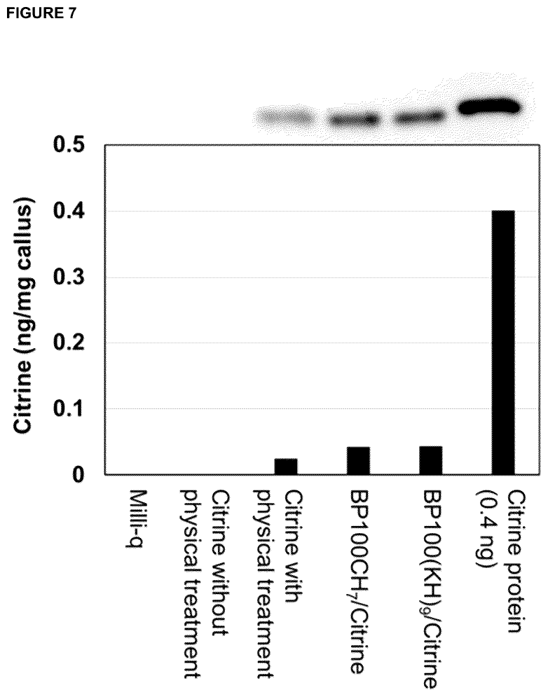

We next quantified the Citrine from the cell extraction by western blot. Samples were prepared by post-infiltration of the BP1 (BP100 (KH)₉-Citrine), BP2 (BP100CH₇-Citrine) and Citrine only with post-infiltration into 5-day callus cell. As controls, the samples with infiltration of Milli-q water and Citrine only without vacuum and pressure treatment were also analyzed (FIG. 7a). The results showed that, besides the BP100 (KH)₉-Citrine and BP100CH₇-Citrine, infiltration of Citrine also exhibited the band of Citrine protein on western blot. Nevertheless, both the results from Milli-q water and Citrine without vacuum and pressure treatment showed no Citrine on western blot. By comparing the area intensity (FIG. 7b) with the positive control, the Citrine delivered without CPP-FP was 0.024 ng/mg callus, delivered by BP100 (KH), was 0.042 ng/mg callus and 0.043 ng/mg callus for BP100CH₇ (FIG. 7c).

By analyzing the fluorescence from Citrine and FM4-64, it is demonstrated that both Citrine-PB100 (KH₉) and Citrine-BP100CH₇ able to pass through the rice cell wall, located on cell membrane and cytoplasm region in rice callus (FIG. 5a-f). This result is in congruence with our following result from FIG. 6a-b. While, compared to the CPP-FP-Citrine complexes, the Citrine without CPP-FP also showed few fluorescence inside of the cell (FIG. 5 g-l, FIG. 6c), however, this result was proved most of the fluorescence comes only from the cell medium layer, only a little among is from the cytoplasm region by the endocytosis of the cell (FIG. 50, FIG. 6). That is because our western blotting result revealed the Citrine without vacuum and pressure treatment is absent (FIG. 7a) on the western blot, suggesting the Citrine in the medium layer was possible introduced by these physical treatments (FIG. 7).

In conclusion, to best of our acknowledge, it is the first time that clear evidence was provided to prove the utility of cell-penetrating peptides BP100 (KH)₉ and BP100CH₇ for protein delivery in rice callus cell. Moreover, our results first demonstrated that 5-day rice callus provides the suitable recipient cells for CPP based molecular delivery. The CPP mediated Citrine delivery process in this study was proposed as the following steps. Firstly, CPP-FP-Citrine complexes were passed through the cell wall into the medium layer of rice callus by a combination physical treatment of vacuum and pressure, then transferred into cells by the interaction between CPP and lipid bilayer. Thus, the fluorescence of Citrine was detected both on the cell membrane and inside of the cells. By contrast, the Citrine without CPP-FP accumulated in medium layer of the cell and only less of them could pass the cell membrane by the endocytosis. This protein delivery system illustrates the possibility for DNA-free genetic modifications in higher plant cells.

### Example 11 Delivery of DNA into Regenerating Rice and Soybean Cells Utilizing Cell Penetrating Peptides

### CPP Preparation

CPP (BP100 KH₉) and dsRed plasmid DNA (Seq ID NO: 1) or Cy3 labelled PNA (peptide nucleic acid) (1:10 molar ratio) were mixed together and incubated for 30 minutes at 4° C. After incubation the volume was made up to 100 μl with water.

Sequence of the cy3 labelled PNA

Cy3-OO-KKK-GTAACAGTTTCTACCTCG-KKK

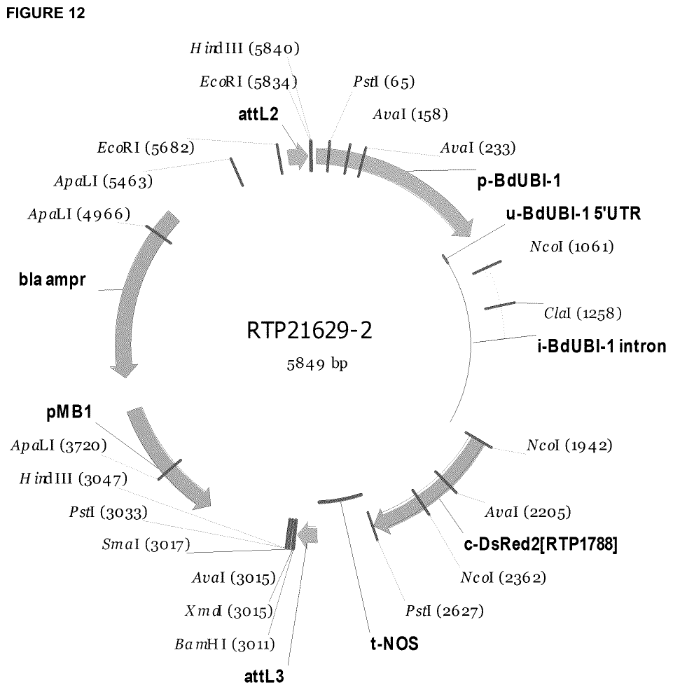

Method for Transformation of dsRed Plasmid DNA (FIG. 12; Seq ID NO: 1) CPP in Rice Callus.

Callus from 5-7 days old rice seeds were used for the experiments. To 10 mg of callus, CPP/DNA or CPP/PNA mixture was added and vacuum infiltrated for 15 mins, washed with distilled water and plated on N6 medium. Expression of dsRed was observed using a scope with a dsRed filter after 4 days. dsRed expression was seen in callus cells.

Method of Transformation of dsRed Plasmid DNA (FIG. 12; Seq ID NO: 1) in Soybean

Primary node from seven days old soybean seedlings were used for the experiments. The explant was submerged in the CPP/DNA or CPP/PNA mixture and vacuum infiltrated for 15 minutes, washed with distilled water and plated on regeneration medium. Expression of dsRed was observed using a scope with a dsRed filter after 4 days.

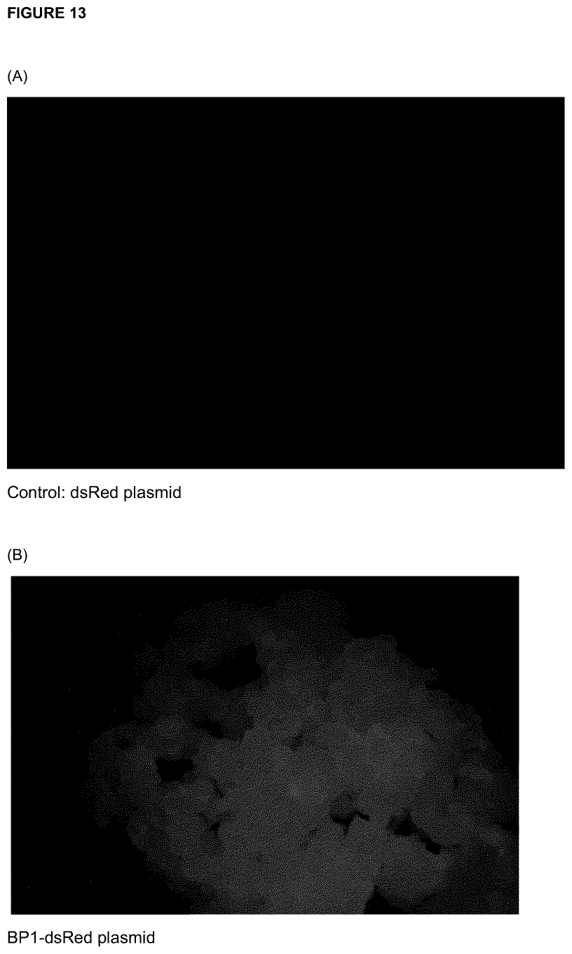

Microscopic analysis of rice callus demonstrated the successful delivery of the dsRed plasmid into regenerating plant cells (FIG. 13). Only in the experiment combining dsRed plasmid DNA (SEQ ID NO: 1) with BP1 cell-penetrating peptide resulted in visible red fluorescence.

### Example 12 Delivery of Cas9 Nuclease into Rice Regenerative Tissue and Genome Editing of Phytoene Desaturase Gene

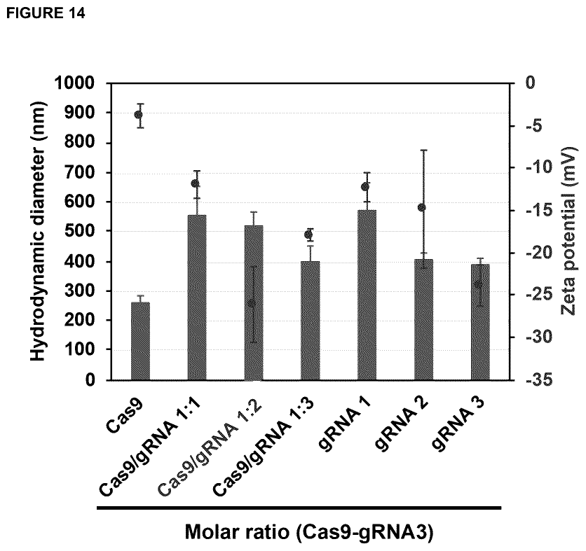

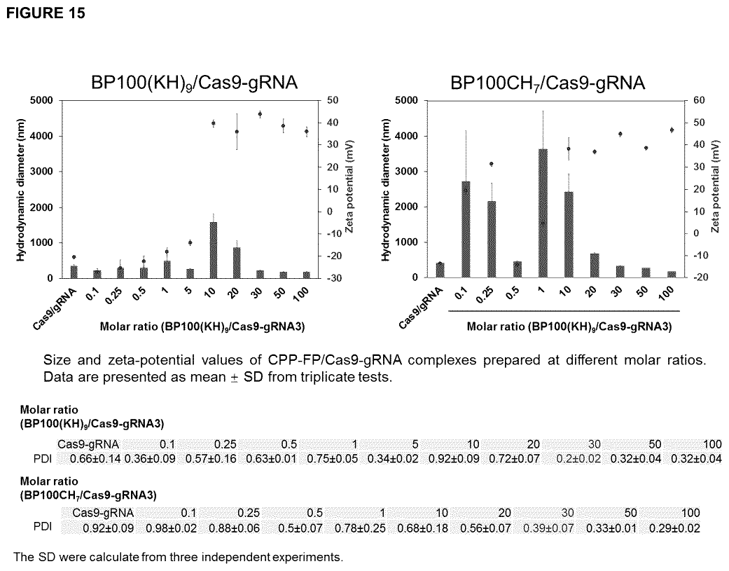

The cell-penetrating peptides BP1 and BP2 were used to bind to SpCas9 protein. 5 different target guideRNAs were used to bind to SpCas9 before BP1 and BP2 were mixed with the nuclease. The guide RNAs were designed to insert mutations into the rice phytoene desaturase gene OsPDS (Seq ID NO: 5), Miki et al. Plant and Cell Physiology 2004:490-495. Mutations in the rice phytoene desaturase result in an albino phenotype (Miki et al. 2004). The guideRNAs Seq ID NO: 6-10 decide in the coding and non-coding sequence of OsPDS. FIG. 14 shows the optimal protein: RNA ratio of 1:2 based on the characterization of the Zeta potential. In a next step, the optimal molar ratio of cell penetration peptides BP1 and BP2 with the Cas9-gRNA complex was tested (FIG. 15). Based on this analysis, an optimal molar ratio of BP1 and BP2 of 30 was used.

For the protein delivery following experimental conditions were applied:
Cas9: 2 μg;
Cas9/gRNA (molar ratio)=0.5
BP100 (KH)₉/Cas9-gRNA=30
BP100CH₇/Cas9-gRNA=30

These components were mixed well at room temperature for 30 minutes.

Then Milli-Q was added to make the solution to 100 μl. This was incubated at room temperature for 30 minutes. The resulting mixture was termed CPP-FP/Cas9-gRNA complex

The CPP-FP/Cas9-gRNA complex (BP1 and BP2 bound to Cas9 with the 5 different gRNAs) was infiltrated into 5 day and 21 day old callus (10 mg callus in 100 μL CPP-FP/Cas9-gRNA complex). The solution was put into a pressure container and either a vacuum of −0.08 MPa for 1 min or pressure of +0.08 MPa for 1 min was applied. After the treatment, callus was washed three times with N6D medium and then transferred on fresh N6D medium (30 g/L lactose, 0.3 g/L casamino acid, 2.8 g/L L-proline, 2 mg/L 2,4-dichlorophenoxyacetic acid (2,4-D), 4.0 g/L CHO (N6) basal salt mix, gelled with 4 g/L phytagel and pH was adjusted to 5.8 before autoclaving).

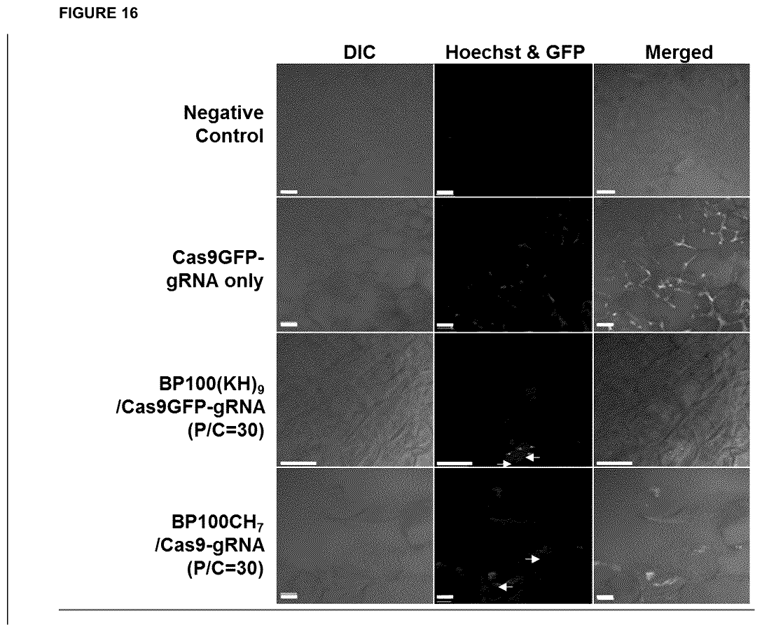

In a parallel experiment to test efficiency of delivery of the CPP-FP/Cas9-gRNA complex into callus, a Cas9-GFP protein was delivered to rice callus cells as described above and visualized using standard Confocal microscopy. Additionally to GFP, Hoechst33342 was used to visualize cell nuclei following standard procedures know in literature. FIG. 16 show the successful delivery of the CPP-FP/Cas9-gRNA complex into the cells and nuclei.

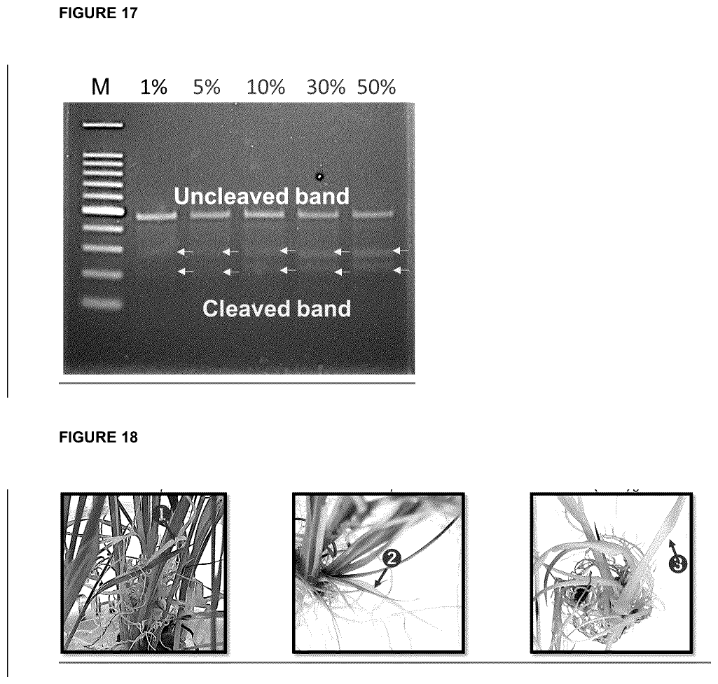

Analysis of regenerating rice callus cells using T7 endonuclease assay demonstrated the successful mutations in OsPDS (FIG. 17) based on delivery of the CPP-FP/Cas9-gRNA complex.

In a next step, plants were regenerated from rice callus as described earlier and analyzed. Based on the publication from Miki (2004), white plant parts or white plants indicate missense mutations in the Ospds gene, indicating successful genome editing of the gene sequence using cell-penetrating peptides as delivery method.

FIG. 18 shows rice plants regenerated from rice callus treated with CPP-FP/Cas9-gRNA complex. For guideRNAs Target 3 and Target 5 white plants/plant parts could be identified (marked by arrows), demonstrating the cell-penetrating peptides were successfully used to achieve non-DNA genome editing.

### Example 13 Characterization of the Binding Part of BP2, the Glutathione-Reducible Peptide KKLFKKILKYLHHCRGHTVHSHHHCIR and its Beneficial Structure

BP2 is a cyclic peptide due to its disulphide bond between the two cysteins. This structure improve binding to proteins, peptides and DNA and improves cell survival (reduced cell toxicity) and regeneration. These advantages of the cyclic structure were analysed by studying a linear, non-cyclic, version of BP2 with regards to its potential to delivery.

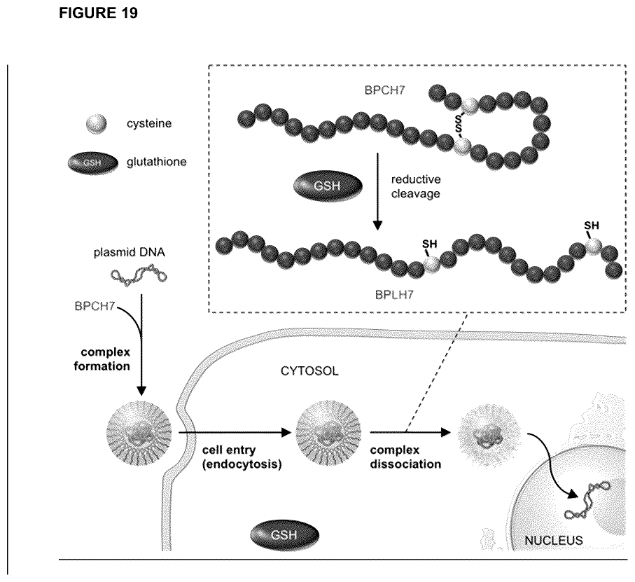

FIG. 19 illustrates the structure of BP2 (BPCH7) and the linear LH7 peptide (BPLH7).

### Experimental Procedures:

Peptides were synthesized using standard 9-fluorenylmethoxycarbonyl (Fmoc) solid phase peptide synthesis. The amino acid sequences and molecular weights are as follows: BPCH7 and BPLH7 (KKLFKKILKYLHHCRGHTVHSHHHCIR, 3,358 Da) BPKH (KKLFKKILKYLKHKHKHKHKHKHKHKHKH, 3,810 Da); BP (KKLFKKILKYL, 1,422 Da); CH₇ (HHCRGHTVHSHHHCIR, 1,954 Da). The purities of these peptides were characterized by HPLC with an Inertsil ODS-3 column (GL Sciences, Tokyo, Japan) at 25° C. (Fig. S1). The mobile phase comprised 15-45% CH₃CN containing 0.1% TFA. The flow rate was 1.0 mL/min.

The pDNA used encoded either Renilla Luciferase (RLuc) or green fluorescent protein (GFP) genes expressed under the control of the constitutive cauliflower mosaic virus 35S promoter (p35S-RLuc-tNOS and p35S-GFP-tNOS, respectively). 17 Glutathione (reduced form) was purchased from Wako Chemical Co. (Osaka, Japan). The DiaEasyTM Dialyzer (1 kDa Molecular Weight Cut-off) was purchased from BioVision, Inc. (Milpitas, CA, USA). The Renilla Luciferase Assay System was purchased from Promega (Madison, WI, USA). The Label IT® Nucleic Acid Labeling Kit, Cy3 was purchased from Mirus Bio, LLC (Madison, WI, USA). Hoechst 33258 and BCECF-AM were purchased from Thermo Fisher Scientific (Waltham, MA, USA).

Formation and Stability of Peptide-pDNA Complexes. Peptide-pDNA complexes were prepared by adding different amounts of each peptide to pDNA at various N/P ratios (0.5, 1, and 2) and autoclaved Milli-Q water to obtain the final volumes required for each experiment. The solution was thoroughly mixed by repeated pipetting and allowed to stabilize for 1, 5, 10, or 24 h at 25° C. Electrophoretic mobility shift assays were performed to detect the stabilities of complexes formed between the peptide and pDNA as previously described. Each peptide was added to pDNA (0.2 μg) at various N/P ratios, adjusted to a final volume of 20 μL, and electrophoresed on a 1% (w/V) agarose gel for 30 min at 100 V.

Extra-/Intracellular pDNA Release and Gene Expression. A time-course study of pDNA release from peptide-pDNA complexes was performed by preparing the complexes at various N/P ratios in Milli-Q water, and then incubating them in phosphate buffer (20 mM, pH 8) supplemented with GSH (10 mM). At 3, 6, 9, 12, 24, and 36 h, complex solutions were loaded on to a 1% (w/V) agarose gel and electrophoresed under the conditions described above. The intensities of selected DNA bands were quantified by densitometry analysis of the gel images using ImageJ software (version 1.48, National Institutes of Health, MD, USA). Released DNA was expressed as the fraction of DNA with restored mobility relative to pDNA alone, based on the relative intensities of the corresponding bands. For intracellular studies, wild-type and transgenic (YFP) A. thaliana plants, which served as model systems in this study, were grown under previously described conditions.¹⁸ Leaves were infiltrated with the complexes by syringe as described, and sampled at the same time points as those used for the time course study of extracellular DNA release. Intracellular expression of the RLuc gene was evaluated quantitatively by the RLuc assay as previously detailed.¹⁷ GFP fluorescence in leaf cells infiltrated with pDNA alone and in complexes with BPLH7 or BPCH7 (N/P 0.5) were observed by CLSM as previously described.¹⁷ Subcellular Localization of Peptide-pDNA Complexes. p35S-GFP-tNOS was labeled with a Nucleic Acid Labeling Kit according to the manufacturer's instructions. Leaves of wild-type or transgenic A. thaliana plants were infiltrated with Cy3pDNA (5 μg or 15 μg) in complex with BPCH7 or BPKH at N/P 0.5 and incubated for 1 h (except for time-lapse experiments). Epidermal and mesophyll cells from the adaxial side were observed and imaged using a CLSM as previously described.¹⁷ Colocalization analysis of micrographs was performed using Zen 2011 operating software. Leaves were stained with 5 μg/ml Hoechst 33258 solution or 10 UM BCECF-AM when needed. Image stacks of transgenic A. thaliana leaves infiltrated with BPCH7-Cy3pDNA or BPKH-Cy3pDNA complexes were collected in the z-direction at 0.3 μm increments for 19.8 μm. 3D reconstructions and digital processing of images and movies were performed using ImageJ software.

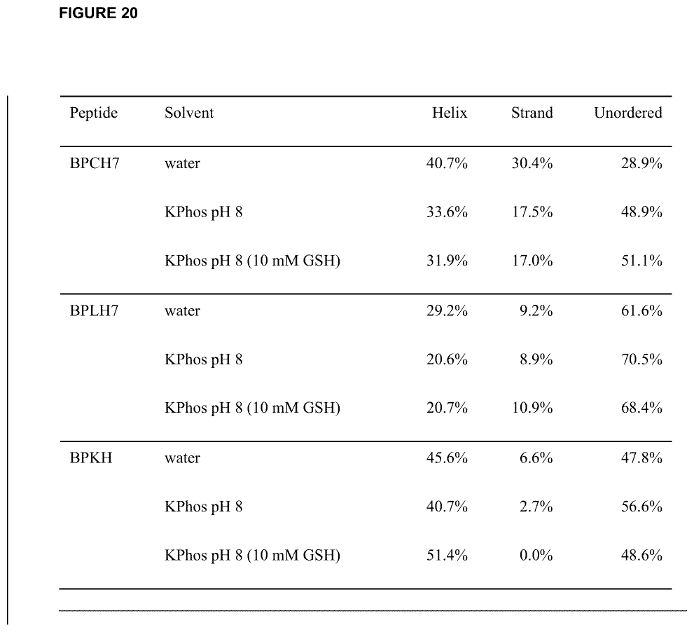

Analysis of secondary structure shows that both BP1 and BP2 have a higher helix and strand order, compared to the higher degree of unordered structure of LH7. This explains the beneficial structure of BP2 (FIG. 20). Also LH7 had significantly lower activity in delivery DNA into rice cells.

Sequences
Seq ID NO: 1 dsRed plasmid DNA; artificial
taaggccacggcggcggcggacacgacggcgacgccccgactccgcgcgcgcgtcaaggctgcagtggcgtcgtggt
ggccgtccgcctgcacgagatccccgcgtggacgagcgccgcctccacccagcccctatatcgagaaatcaacggtg
ggctcgagctcctcagcaacctccccacccccccttccgaccacgctcccttcccccgtgcccctcttctccgtaaa
cccgagccgccgagaacaacaccaacgaaagggcgaagagaatcgccatagagaggagatgggcggaggcggatagt
ttcagccattcacggagaaatggggaggagagaacacgacatcatacggacgcgaccctctagctggctggctgtcc
taaagaatcgaacggaatcgctgcgccaggagaaaacgaacggtcctgaagcatgtgcgcccggttcttccaaaaca
cttatctttaagattgaagtagtatatatgactgaaatttttacaaggtttttccccataaaacaggtgagcttatc
tcatccttttgtttaggatgtacgtattatatatgactgaatattttttattttcattgaatgaagattttcgaccc
cccaaaaataaaaaacggagggagtacctttgtgccgtgtatatggactagagccatcgggacgtttccggagactg
cgtggtgggggcgatggacgcacaacgaccgcattttcggttgccgactcgccgttcgcatctggtaggcacgactc
gtcgggttcggctcttgcgtgagccgtgacgtaacagacccgttctcttcccccgtctggccatccataaatccccc
ctccatcggcttccctttcctcaatccagcaccctgattccgatcgaaaagtccccgcaagagcaagcgaccgatct
cgtgaatctccgtcaaggtatgcagcctcgcttcctcctcgctaccgtttcaattctggagtaggtcgtagaggata
ccatgttgatttgacagagggagtagattagatacttgtagatcgaagtgcggatgttccatggtagatgataccat
gttgatttcgattagatcggattaaatctttgtagatcgaagtgcgcatgttccatgaattgcctgttaccagtaga
ttcaagtttttctgtgttatagaggtgggatctactcgttgagatgattagctcctagaggacaccatgccgttttg
gaaaatagatcagaaccgtgtagatcgatgtgagcatgtgttcctgtagatccaagttctttcgcatgttactagtt
gtgatctattgtttgtgtaatacgctctcgatctatccgtgtagatttcactcgattactgttactgtggcttgatc
gttcatagttgttcgttaggtttgatcgaacagtgtctgaacctaattggatatgtattcttgatctatcaacgtgt
aggtttcagtcatgtatttatgtactccctccgtcccaaattaactgacgtggattttgtataagaatctatacaaa
tccatgtcagttaattcgggatggagtaccatattcaataatttgtttattgctgtccacttatgtaccatatgttt
gttgttcctcatgtggattctactaattatcattgattggtgatcttctattttgctagtttcctagctcaatctgg
ttattcatgtagatgtgttgttgaaatcggagaccatgcttgttattagatagtttattgcttatcagtttcatgtt
ctggttgatgcaacacatattcatgttcgctatctggttgctgcttgatattctctgatttacattcattataagaa
tatattctgctctggttgttgcttctcatgactttacctactcggtaggtgacttaccttttggtttacaattgtca
actatgcagggcgcgccatggcctcctccgagaacgtcatcaccgagttcatgcgcttcaaggtgcgcatggagggc
accgtgaacggccacgagttcgagatcgagggcgagggcgagggccgcccctacgagggccacaacaccgtgaagct
gaaggtgaccaagggcggccccctgcccttcgcctgggacatcctgtccccccagttccagtacggctccaaggtgt
acgtgaagcaccccgccgacatccccgactacaagaagctgtccttccccgagggcttcaagtgggagcgcgtgatg
aacttcgaggacggcggcgtggcgaccgtgacccaggactcctccctccaggacggctgcttcatctacaaggtgaa
gttcatcggcgtgaacttcccctccgacggccccgtgatgcagaagaagaccatgggctgggaggcctccaccgagc
gcctgtacccccgcgacggcgtgctgaagggcgagacccacaaggccctgaagctgaaggacggcggccactacctg
gtggagttcaagtccatctacatggccaagaagcccgtgcagctgcccggctactactacgtggacgccaagctgga
catcacctcccacaacgaggactacaccatcgtggagcagtacgagcgcaccgagggccgccaccacctgttcctgt
agcctgcaggcctaggatcgttcaaacatttggcaataaagtttcttaagattgaatcctgttgccggtcttgcgat
gattatcatataatttctgttgaattacgttaagcatgtaataattaacatgtaatgcatgacgttatttatgagat
gggtttttatgattagagtcccgcaattatacatttaatacgcgatagaaaacaaaatatagcgcgcaaactaggat
aaattatcgcgcgcggtgtcatctatgttactagatcggccggccgtttaaaccaactttattatacaaagttggca
ttataaaaaagcattgcttatcaatttgttgcaacgaacaggtcactatcagtcaaaataaaatcattatttgaccc
aatatcggatcccgggcccgtcgactgcagaggcctgcatgcaagcttggcgtaatcatggtcatagctgtttcctg
tgtgaaattgttatccgctcacaattccacacaacatacgagccggaagcataaagtgtaaagcctggggtgcctaa
tgagtgagctaactcacattaattgcgttgcgctcactgcccgctttccagtcgggaaacctgtcgtgccagctgca
ttaatgaatcggccaacgcgcggggagaggcggtttgcgtattgggcgctcttccgcttcctcgctcactgactcgc
tgcgctcggtcgttcggctgcggcgagcggtatcagctcactcaaaggcggtaatacggttatccacagaatcaggg
gataacgcaggaaagaacatgtgagcaaaaggccagcaaaaggccaggaaccgtaaaaaggccgcgttgctggcgtt
tttccataggctccgcccccctgacgagcatcacaaaaatcgacgctcaagtcagaggtggcgaaacccgacaggac
tataaagataccaggcgtttccccctggaagctccctcgtgcgctctcctgttccgaccctgccgcttaccggatac
ctgtccgcctttctcccttcgggaagcgtggcgctttctcatagctcacgctgtaggtatctcagttcggtgtaggt
cgttcgctccaagctgggctgtgtgcacgaaccccccgttcagcccgaccgctgcgccttatccggtaactatcgtc
ttgagtccaacccggtaagacacgacttatcgccactggcagcagccactggtaacaggattagcagagcgaggtat
gtaggcggtgctacagagttcttgaagtggtggcctaactacggctacactagaagaacagtatttggtatctgcgc
tctgctgaagccagttaccttcggaaaaagagttggtagctcttgatccggcaaacaaaccaccgctggtagcggtg
gtttttttgtttgcaagcagcagattacgcgcagaaaaaaaggatctcaagaagatcctttgatcttttctacgggg
tctgacgctcagtggaacgaaaactcacgttaagggattttggtcatgagattatcaaaaaggatcttcacctagat
ccttttaaattaaaaatgaagttttaaatcaatctaaagtatatatgagtaaacttggtctgacagttaccaatgct
taatcagtgaggcacctatctcagcgatctgtctatttcgttcatccatagttgcctgactccccgtcgtgtagata
actacgatacgggagggcttaccatctggccccagtgctgcaatgataccgcgagacccacgctcaccggctccaga
tttatcagcaataaaccagccagccggaagggccgagcgcagaagtggtcctgcaactttatccgcctccatccagt
ctattaattgttgccgggaagctagagtaagtagttcgccagttaatagtttgcgcaacgttgttgccattgctaca
ggcatcgtggtgtcacgctcgtcgtttggtatggcttcattcagctccggttcccaacgatcaaggcgagttacatg
atcccccatgttgtgcaaaaaagcggttagctccttcggtcctccgatcgttgtcagaagtaagttggccgcagtgt
tatcactcatggttatggcagcactgcataattctcttactgtcatgccatccgtaagatgcttttctgtgactggt
gagtactcaaccaagtcattctgagaatagtgtatgcggcgaccgagttgctcttgcccggcgtcaatacgggataa
taccgcgccacatagcagaactttaaaagtgctcatcattggaaaacgttcttcggggcgaaaactctcaaggatct
taccgctgttgagatccagttcgatgtaacccactcgtgcacccaactgatcttcagcatcttttactttcaccagc
gtttctgggtgagcaaaaacaggaaggcaaaatgccgcaaaaaagggaataagggcgacacggaaatgttgaatact
catactcttcctttttcaatattattgaagcatttatcagggttattgtctcatgagcggatacatatttgaatgta
tttagaaaaataaacaaataggggttccgcgcacatttccccgaaaagtgccacctgacgtctaagaaaccattatt
atcatgacattaacctataaaaataggcgtatcacgaggccctttcgtctcgcgcgtttcggtgatgacggtgaaaa
cctctgacacatgcagctcccggagacggtcacagcttgtctgtaagcggatgccgggagcagacaagcccgtcagg
gcgcgtcagcgggtgttggcgggtgtcggggctggcttaactatgcggcatcagagcagattgtactgagagtgcac
catatgcggtgtgaaataccgcacagatgcgtaaggagaaaataccgcatcaggcgccattcgccattcaggctgcg
caactgttgggaagggcgatcggtgcgggcctcttcgctattacgccagctggcgaaagggggatgtgctgcaaggc
gattaagttgggtaacgccagggttttcccagtcacgacgttgtaaaacgacggccagtgaattcgagctcggtacc
tcgcgaatgcatctagatgacccaatcaaataatgattttattttgactgatagtgacctgttcgttgcaacaaatt
gataagcaatgctttcttataatgccaactttgtacaagaaagctgggtatttaaatgaattcaagcttttaat
Seq ID NO 2: BP1, amino acid sequence, synthetic
KKLFKKI LKYLKHKHKHKHKHKHKHKHKH
Seq ID NO 3: BP2, amino acid sequence, synthetic
KKLFKKI LKYLHHCRGHTVHSHHHCIR
Seq ID NO 4: BP3, amino acid sequence, synthetic
KKLFKKILKYLKKLFKKILKYLKKKKKKKK
>Seq ID NO 5: OsPDS DNA rice
CATCTTCCACAATCCTCACCCCCGCCTCCCCTTTGTCCCTTTCCCACCGCCCCAAAAACC
CACCCCCTCCCTGACTCCTCCCCCCGCAGCTTCCGCCGTCCGCCTCCGCTCCCACGTCGC
CGCCCGCTCGTCGTCGCCGCCGGTGAGTCTCCTCCACTCCGTGCTCGCCCCCTCCGTACC
CAGCAGCAGGATCGGATCGGTCGCGCGGGCGGCGGGGGTACGTATCTGTATTCCGTAGAA
TTGGGGGAATTCATTCCGGGTTGCGGGGTTGCTAAGGTGTTGGATTGACTGCGGTGACGG
GAGGGCGGTAGTTTCCTGGTAAATAGGTAGTAGGAGAGATGCTGAGATGACTGCTGGCTT
TGAGCATGCGGCATATGATGATTTAGTGCTTAGTTTGGGGGCTTATCTTTAGATACTAGC
GGGCGCATGGTTGTGAGTTCAGTTTGCGCTAACCACCACTTTTGCATGAGGAGGCAAACG
AGGTCCTCTCCAGCTGCCCTGCCCTAGTGTGATATCATTTGAGCCTTTCATGCTTTTTGT
GCCATGCTTGATCTGTTCCAATCCATTTACTTCACTAACCAAATTATGCGGGTCATATGC
AGTTTTCCTTTCATGTTTCTCTCCAACTATAAAAGTTTGATTGGCCGCAGCCACATAGAG
AAACTCGGAAGATTAGGGAGTAAACCAATATTACCACTGTCCACATAGCTTTAACAACTA
ACAGCTGGTCCTGCTCTTTTTTTCCTTTTGGCATCAGTTTGTTATTGTCATGCTATGTTT
CCATTTGACGACTGGACTAGAATAGAATCTGTTTCTTTGGCTCGTTTTTTTTTTTTCATC
AAATAGTGATGACAAACTTGATAAATTTACATACTGATACAGTGATACTTGGCTGACTTT
CATAACAAACGGTTTTGTGTATTGTGTGTTTAATGGTTCCTCTTGTTTTTGCAGACGCTC
TTGCGTGCTTATTTGTCAAATCAGATCTGAATATAATTTTAGGAGTTGCTTCAGCATGGA
TACTGGCTGCCTGTCATCTATGAACATAACTGGAACCAGCCAAGCAAGATCTTTTGCGGG
ACAACTTCCTACTCATAGGTGCTTCGCAAGTAGCAGCATCCAAGCACTGAAAAGTAGTCA
GCATGTGAGCTTTGGAGTGAAATCTCTTGTCTTAAGGAATAAAGGAAAAAGATTCCGTCG
GAGGCTCGGTGCTCTACAGGTTCAACCTTTGTACTCTATTATTGCCTCACATTCCATCTC
TTGTGAAAATATATTTGATTGGCTTTTCTGCAGGTTGTTTGCCAGGACTTTCCAAGACCT
CCACTAGAAAACACAATAAACTTTTTGGAAGCTGGACAACTATCTTCATTTTTCAGAAAC
AGTGAACAACCCACTAAACCATTACAGGTCGTGATTGCTGGAGCAGGTATGATATAATTC
TAGGATTTGACAGATGAATAATTTACATATATATCTAACTTTGATAGCAGTCACATCGTG
GTCTTAGCATTGTAGTTTTTAGCTTTGATTTTTTTTTCAGGATTAGCTGGTTTATCAACG
GCAAAATATCTGGCAGATGCTGGTCATAAACCCATATTGCTTGAGGCAAGGGATGTTTTG
GGTGGAAAGGTTTTACTCTTATGCTTTTATGTTGCATTTAATTTTTTTTGTTATTCATTC
TTTTTTTTTTTGGTTGCCTTTATCTTAATAGCTCATATTCACTGTTAGTAGCATTTGTGG
ATTATTGTTTTTTTTTTTGGGGAAATGCCTTGAACAGATAGCTGCTTGGAAGGATGAAGA
TGGAGATTGGTATGAAACTGGGCTTCATATCTTTTGTAAGTAATAACTCTGGATTTTTAA
GGTTCTCGTTGTGCTATATTTTATTTAGGTTATTACCGCCAGCACTGATAGATATCTCTA
AGGGTTTTGAACAAAAAAACATGTATCAAACTCTTTCATCGATAAGGTAGAAATGCCATG
CGGGAAGTATGAAGTGATGTCTGAGGATTAACACACATGGTAGTTTTATTTTGTAAGAAA
CTTTTAGATTGGTTTTTTTCACAGTACTAAAAAGTAACTTTTTACTAGCTTATATGGTTG
ATAAATTTTAACGTCACATAAATATCATGAGCTAATTGAATATAAATCCTCCTGTTCATA
CATAGTCTTCTTTCAACCTACTATTCCCTTCCAAACATATATGAATATGACAGATACTGT
TTTTCCTTCCATGCTCACACTGTTTTGTCGTCCACAACAGTACATATGTGACATTGTTCA
TTTTGTGCCTGTATGTAACCATATACCTTTTTGGTTTAAGTTGGAGCTTATCCCAACATA
CAGAACTTGTTTGGCGAGCTTGGTATTAATGATCGGTTGCAATGGAAGGAACACTCCATG
ATATTTGCCATGCCAAACAAGCCAGGAGAATTCAGCCGGTTTGATTTTCCTGAAACATTG
CCTGCACCCTTAAATGGTGAGATCATATGCAGCGCTGGAGTTGTTTAATTAAACCAAGAT
TCCCAGAAGTACATCGTATTGGTGGTTACTTTTGTTTTACTAACACATGACTGTAATTAG
GGGGTATATTACTAGCAACGTTAATGATAGATCAATAGATCATGCCATGGAGCTTTTATG
TTGTCAATTGATGCCTATTTATTATTTATCATTGATCATGCGTGCATTTAACAGGAATAT
GGGCCATACTAAGAAACAATGAAATGCTAACTTGGCCAGAGAAGGTGAAGTTTGCTCTTG
GACTTTTGCCAGCAATGGTTGGTGGCCAAGCTTATGTTGAAGCTCAAGATGGTTTTACTG
TTTCTGAGTGGATGAAAAAGCAGGTATAAGTTCACAATATCAGTTTGTCAAGTCTCTGTG
TACAAGACACATTTCTACCTCATTAATTTGGAATGGATATAGGAGAAGGTGTTGTAAGCT
AGAAAACCTTTTATTTTCTAATAAAAAAACTGATGCCCTTTATTGTTGCATTCACATTGG
GAAGAACTGGCAGTTCTGAGGATGAAATGCTTCATGTACTCAAGTTTATGCCCTTTATTT
TGCCCAGATCCTTTTGCACAGGTTTAAGCTTGAGCTATGCTTTTAGTTTAAGACCACTGT
TTCAGTTAAAGGTCAACAACCTTGCATGATTTCTTCCTCCACCTAGAAAAGCCATTGCAC
ATATTGACAAAGCACACAATCCTGTTGACTATATTCTTTATGAGCTAATATACAGAACTG
TTTTATACAGAAAACACAATACATATGCTATAGTTATCAATCTCTTTCCCTTTTTTTGGG
ATAACGGATTAATATGGTGCCTGATACAGTTGTTTGATCAGCACAGGGTGTTCCTGATCG
AGTGAACGATGAGGTTTTCATTGCAATGTCAAAGGCACTTAATTTCATAAATCCTGATGA
GTTATCCATGCAGTGCATTCTGATTGCTTTAAACCGATTTCTTCAGGTATTTATTATGTT
GCTCTATGGTCATGTGTGTTGCATATGAGTAATTCTTCTGTTCTTTCCGGAGTAGTACCT
TACGTATTACATCCTTCTTAGTGTTTCTTGTCTCTGTTGTTTCCTACCTTGAGGAAACTC
AAATGAATTTTCGCTTAGAGGCCTTTTAAAAAAAATTATGCAAATGTGTAGGAGAAGCAT
GGTTCTAAGATGGCATTCTTGGATGGTAATCCTCCTGAAAGGTTATGCATGCCTATTGTT
GACCATGTTCGCTCTTTGGGTGGTGAGGTTCGGCTGAATTCTCGTATTCAGAAAATAGAA
CTTAATCCTGATGGAACAGTGAAACACTTTGCACTTACTGATGGAACTCAAATAACTGGA
GATGCTTATGTTTTTGCAACACCAGGTGATTTTCTACAATCTTTGTTTCTTCTGCAGTTC
ATAAATTATATATATGCGGCTACTCATTTTAACTGACTAGCCTGTATTTAGTTGATATCT
TGAAGCTTCTTGTACCTCAAGAGTGGAAAGAAATATCTTATTTCAAGAAGCTGGAGAAGT
TGGTGGGAGTTCCTGTTATAAATGTTCATATATGGTTGGTTGGTTGAATTATTTGGTTCC
AAGTCGGAAATTACTCATCATCGAGTTTGTGGTTCTCCTTATGACTCATATTAGTATTTC
TGTTGGTTTGAACATTTCAGGTTTGATAGAAAACTGAAGAACACATATGACCACCTTCTT
TTCAGCAGGTGTCTCTTCTAATTCCTCATCAGTTTTGCTGTCCTTTCACTGCCTCATGCA
TTTGCTCTGTGCTATGACTGGTTTATGAACTAAAACGATTTGTATTGCCCAAATTGGGCA
CATTCTATCCTGATTTTGTATACATTCTTGATTAATACCAAATATCATATGTCCCATGTA
TTGATCTTGTTCCCTTTTCTTTCAGGAGTTCACTTTTAAGTGTTTATGCGGACATGTCAG
TAACTTGCAAGGTACTAACTAGGAGACATTATATGTTACGAAATAGTAACTATCTGTCAT
GTATTATTGCTCTTGTGTATTTGTTCTTGGGTTTACCATCTTCAAGCATCACATGATATT
TATTTTAGTAGCTGTAACAAAAGGCCCAAAAGTGCATGTGTTACAGAAGGAATCCAGTAT
TAATTATTAAACTTGGAAAGTAGATATATTTTATTTCAGATTCATTTAGGCAACATGTCA
CTTGGCTCTAGAGTCTAGATTTTATGGACCATAATAGCTCAGGAAATTAAAGACATGGAT
GCCTACTGAACGGTTTTCTTTCCTTTTGTTTTGAACTCTTTACAGGAATACTATGATCCA
AACCGTTCAATGCTGGAGTTGGTCTTTGCTCCTGCAGAGGAATGGGTTGGACGGAGTGAC
ACTGAAATCATCGAAGCAACTATGCAAGAGCTAGCCAAGCTATTTCCTGATGAAATTGCT
GCTGATCAGAGTAAAGCAAAGATTCTGAAGTATCATGTTGTGAAGACACCAAGGTGAGGA
CATTTTGCAAGAGCGCCCCCTATCTGATATATCATAGGTAGGTCTAATAGTTGGATGCAC
ACTTCTCTCACGTTCCTTTCTTTTCTGTCTCACTGTTACAGATCTGTTTACAAGACTATC
CCGGACTGTGAACCTTGCCGACCTCTGCAAAGATCACCGATTGAAGGGTTCTATCTAGCT
GGTGACTACACAAAGCAGAAATATTTGGCTTCGATGGAGGGTGCAGTTCTATCTGGGAAG
CTTTGTGCTCAGTCTGTAGTGGAGGTAAACGCTGCTCTCCATGGTTCTGTTTGTACATAG
ATGCATCAGACTTGTATTGTTGTCTTGGTGCAGTTCACAATGATTCAGTTTTGTAGGCTA
ATGAGTTATCACTTGCTGATTTCAGGATTATAAAATGCTATCTCGTAGGAGCCTGAAAAG
TCTGCAGTCTGAAGTTCCTGTTGCCTCCTAGTTGTAGTCAGGACTATTCCCAATGGTGTG
TGTGTCATCATCCCCTAGTCAGTTTTTTTCTATTTAGTGGGTGCCCAACTCTCCACCAAT
TTACACATGATGGAACTTGAAAGATGCCTATTTTGGTCTTATCATATTTCTGTAAAGTTG
ATTTGTGACTGAGAGCTGATGCCGATATGCCATGCTGGAGAAAAAGAACATTATGTAAAA
CGACCTGCATAGTAATTCTTAGACTTTTGCAAAAGGCAAAAGGGGTAAAGCGACCTTTTT
TTTCTATGTGAAGGGATTAAGAGACCTTA
>Seq ID NO 6: OsPDS_target_1; DNA synthetic
GTTGGTCTTTGCTCCTGCAGAGG
>Seq ID NO 7: OsPDS_target_2; DNA synthetic
CCTGCAGAGGAATGGGTTGGAC
>Seq ID NO 8: OsPDS_target_3; DNA synthetic
CCTGTTATAAATGTTCATATATG
>Seq ID NO 9: OsPDS_target_4; DNA synthetic
CCTTACGTATTACATCCTTCTTA
>Seq ID NO 10: OsPDS_target_5; DNA synthetic
ACAGTTGTTTGATCAGCACAGGG
SEQ ID NO: 11: Cell penetrating peptide sequence.
KKLFKKILKYL
SEQ ID NO: 12: Polycation sequence
HHCRGHTVHSHHHCIR

1. A complex comprising a first component: (i) a carrier peptide comprising a cell-penetrating sequence and a polycation sequence: and a second component (ii) a ribonucleic acid (RNA), PNA and/or protein, wherein the carrier peptide is a cyclic peptide comprising at least 2 cysteine residues bridged by a disulphide bond.

2. The complex of claim 1, wherein the carrier peptide is that defined in SEQ ID 3.

3. The complex of claim 1, wherein component (ii) comprises a protein, and the protein is a nuclease, a TALEN, peptide nucleic acid or a zinc finger transcription factor.

4. The complex of claim 3, wherein the nuclease is a RNA guided nuclease.

5. The complex of claim 4, wherein the RNA guided nuclease is Cas9.

6. The complex of claim 1, wherein the RNA is a guide RNA.

7. The complex of claim 1, wherein component (ii) comprises Cas9 and a guide RNA.

8. The complex of claim 1, wherein the molar ratio of the carrier peptide to component (ii) is between 1:1 and 100:1.

9. A method of preparing a complex of claim 1, comprising
(i) preparing a sample of the carrier peptide component;
(ii) preparing a sample of the ribonucleic acid (RNA), PNA and/or protein component;
(iii) mixing samples (i) and (ii) at room temperature; and
(iv) allowing the resulting solution to incubate for 30 mins to 60 mins in the dark; wherein the molar ratio of the carrier peptide to component (ii) is between 1:1 and 100:1.

10. A method of introducing ribonucleic acid, PNA and/or protein to a target plant cell(s), comprising the step of bringing the complex of claim 1 into contact with the target plant cell(s).

11. The method of claim 10, wherein the target plant cell is selected from the group comprising tobacco, carrot, maize, canola, rapeseed, cotton, palm, peanut, soybean, sunflower, wheat, Oryza sp., Arabidopsis sp., Ricinus sp., and sugarcane.

12. The method of claim 10, wherein the plant cell is from a tissue selected from the group consisting of embryo, meristematic, callus, explant, seedlings, pollen, leaves, anthers, roots, root tips, flowers, seeds, pods and stems.

13. The method of claim 12, wherein the plant cell is rice callus tissue, and wherein the complex is brought into contact with the callus tissue by incubating the callus tissue with the complex at −0.08 MPa for 1 min, then incubating the callus tissue with the complex at +0.08 MPa for 1 min, then incubating the callus tissue at 30° C. in the dark.

14. The method of claim 12, wherein the plant cell is soybean explant tissue, and wherein the complex is brought into contact with the soybean explant tissue by vacuum infiltration.

15. A method effecting a genetic alteration in the genome of a plant cell comprising: (i) exposing the plant, or a tissue, cell or callus of a plant, to the complex defined in claim 1,
wherein component (ii) of the complex comprises (a) an RNA-guided nuclease, and (b) at least one guide RNA or polynucleotide encoding a guide RNA;
wherein the at least one guide RNA is capable of directing the RNA-guided nuclease to a defined location in the genome, thereby effecting a genetic alteration at the defined location in the genome; and
wherein the genetic alteration is at least one alteration selected from the group consisting of insertion of at least one nucleotide, deletion of at least one nucleotide, or replacement of at least one nucleotide at the defined location in the genome or any combination thereof.

16. The method of claim 15, wherein the RNA-guided nuclease is Cas9.

17. The method of claim 15, wherein the ratio of (a) the RNA-guided nuclease, and (b) at least one guide RNA is 0.5.

18. The method of claim 13, wherein the molar charge of the carrier peptide to component (ii) is 30:1.


SEQUENCE LISTING (12 sequences)

SEQ ID NO: 1 - DNA, 5849 bp, Artificial sequence
  dsRed plasmid DNA
taaggccacggcggcggcggacacgacggcgacgccccgactccgcgcgcgcgtcaaggctgcagtggcgtcgtggtggccgtccgcctgcacgagatccccgcgtggacgagcgccgcctccacccagcccctatatcgagaaatcaacggtgggctcgagctcctcagcaacctccccacccccccttccgaccacgctcccttcccccgtgcccctcttctccgtaaacccgagccgccgagaacaacaccaacgaaagggcgaagagaatcgccatagagaggagatgggcggaggcggatagtttcagccattcacggagaaatggggaggagagaacacgacatcatacggacgcgaccctctagctggctggctgtcctaaagaatcgaacggaatcgctgcgccaggagaaaacgaacggtcctgaagcatgtgcgcccggttcttccaaaacacttatctttaagattgaagtagtatatatgactgaaatttttacaaggtttttccccataaaacaggtgagcttatctcatccttttgtttaggatgtacgtattatatatgactgaatattttttattttcattgaatgaagattttcgaccccccaaaaataaaaaacggagggagtacctttgtgccgtgtatatggactagagccatcgggacgtttccggagactgcgtggtgggggcgatggacgcacaacgaccgcattttcggttgccgactcgccgttcgcatctggtaggcacgactcgtcgggttcggctcttgcgtgagccgtgacgtaacagacccgttctcttcccccgtctggccatccataaatcccccctccatcggcttccctttcctcaatccagcaccctgattccgatcgaaaagtccccgcaagagcaagcgaccgatctcgtgaatctccgtcaaggtatgcagcctcgcttcctcctcgctaccgtttcaattctggagtaggtcgtagaggataccatgttgatttgacagagggagtagattagatacttgtagatcgaagtgcggatgttccatggtagatgataccatgttgatttcgattagatcggattaaatctttgtagatcgaagtgcgcatgttccatgaattgcctgttaccagtagattcaagtttttctgtgttatagaggtgggatctactcgttgagatgattagctcctagaggacaccatgccgttttggaaaatagatcagaaccgtgtagatcgatgtgagcatgtgttcctgtagatccaagttctttcgcatgttactagttgtgatctattgtttgtgtaatacgctctcgatctatccgtgtagatttcactcgattactgttactgtggcttgatcgttcatagttgttcgttaggtttgatcgaacagtgtctgaacctaattggatatgtattcttgatctatcaacgtgtaggtttcagtcatgtatttatgtactccctccgtcccaaattaactgacgtggattttgtataagaatctatacaaatccatgtcagttaattcgggatggagtaccatattcaataatttgtttattgctgtccacttatgtaccatatgtttgttgttcctcatgtggattctactaattatcattgattggtgatcttctattttgctagtttcctagctcaatctggttattcatgtagatgtgttgttgaaatcggagaccatgcttgttattagatagtttattgcttatcagtttcatgttctggttgatgcaacacatattcatgttcgctatctggttgctgcttgatattctctgatttacattcattataagaatatattctgctctggttgttgcttctcatgactttacctactcggtaggtgacttaccttttggtttacaattgtcaactatgcagggcgcgccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctccaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcctgcaggcctaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggccggccgtttaaaccaactttattatacaaagttggcattataaaaaagcattgcttatcaatttgttgcaacgaacaggtcactatcagtcaaaataaaatcattatttgacccaatatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgacccaatcaaataatgattttattttgactgatagtgacctgttcgttgcaacaaattgataagcaatgctttcttataatgccaactttgtacaagaaagctgggtatttaaatgaattcaagcttttaat

SEQ ID NO: 2 - protein, 29 aa, Artificial sequence
  BP1 amino acid sequence, synthetic
KKLFKKILKYLKHKHKHKHKHKHKHKHKH

SEQ ID NO: 3 - protein, 27 aa, Artificial sequence
  BP2 amino acid sequence, synthetic
KKLFKKILKYLHHCRGHTVHSHHHCIR

SEQ ID NO: 4 - protein, 30 aa, Artificial sequence
  BP3 amino acid sequence, synthetic
KKLFKKILKYLKKLFKKILKYLKKKKKKKK

SEQ ID NO: 5 - DNA, 5729 bp, Oryza sp.
catcttccacaatcctcacccccgcctcccctttgtccctttcccaccgccccaaaaacccaccccctccctgactcctccccccgcagcttccgccgtccgcctccgctcccacgtcgccgcccgctcgtcgtcgccgccggtgagtctcctccactccgtgctcgccccctccgtacccagcagcaggatcggatcggtcgcgcgggcggcgggggtacgtatctgtattccgtagaattgggggaattcattccgggttgcggggttgctaaggtgttggattgactgcggtgacgggagggcggtagtttcctggtaaataggtagtaggagagatgctgagatgactgctggctttgagcatgcggcatatgatgatttagtgcttagtttgggggcttatctttagatactagcgggcgcatggttgtgagttcagtttgcgctaaccaccacttttgcatgaggaggcaaacgaggtcctctccagctgccctgccctagtgtgatatcatttgagcctttcatgctttttgtgccatgcttgatctgttccaatccatttacttcactaaccaaattatgcgggtcatatgcagttttcctttcatgtttctctccaactataaaagtttgattggccgcagccacatagagaaactcggaagattagggagtaaaccaatattaccactgtccacatagctttaacaactaacagctggtcctgctctttttttccttttggcatcagtttgttattgtcatgctatgtttccatttgacgactggactagaatagaatctgtttctttggctcgttttttttttttcatcaaatagtgatgacaaacttgataaatttacatactgatacagtgatacttggctgactttcataacaaacggttttgtgtattgtgtgtttaatggttcctcttgtttttgcagacgctcttgcgtgcttatttgtcaaatcagatctgaatataattttaggagttgcttcagcatggatactggctgcctgtcatctatgaacataactggaaccagccaagcaagatcttttgcgggacaacttcctactcataggtgcttcgcaagtagcagcatccaagcactgaaaagtagtcagcatgtgagctttggagtgaaatctcttgtcttaaggaataaaggaaaaagattccgtcggaggctcggtgctctacaggttcaacctttgtactctattattgcctcacattccatctcttgtgaaaatatatttgattggcttttctgcaggttgtttgccaggactttccaagacctccactagaaaacacaataaactttttggaagctggacaactatcttcatttttcagaaacagtgaacaacccactaaaccattacaggtcgtgattgctggagcaggtatgatataattctaggatttgacagatgaataatttacatatatatctaactttgatagcagtcacatcgtggtcttagcattgtagtttttagctttgatttttttttcaggattagctggtttatcaacggcaaaatatctggcagatgctggtcataaacccatattgcttgaggcaagggatgttttgggtggaaaggttttactcttatgcttttatgttgcatttaattttttttgttattcattctttttttttttggttgcctttatcttaatagctcatattcactgttagtagcatttgtggattattgtttttttttttggggaaatgccttgaacagatagctgcttggaaggatgaagatggagattggtatgaaactgggcttcatatcttttgtaagtaataactctggatttttaaggttctcgttgtgctatattttatttaggttattaccgccagcactgatagatatctctaagggttttgaacaaaaaaacatgtatcaaactctttcatcgataaggtagaaatgccatgcgggaagtatgaagtgatgtctgaggattaacacacatggtagttttattttgtaagaaacttttagattggtttttttcacagtactaaaaagtaactttttactagcttatatggttgataaattttaacgtcacataaatatcatgagctaattgaatataaatcctcctgttcatacatagtcttctttcaacctactattcccttccaaacatatatgaatatgacagatactgtttttccttccatgctcacactgttttgtcgtccacaacagtacatatgtgacattgttcattttgtgcctgtatgtaaccatatacctttttggtttaagttggagcttatcccaacatacagaacttgtttggcgagcttggtattaatgatcggttgcaatggaaggaacactccatgatatttgccatgccaaacaagccaggagaattcagccggtttgattttcctgaaacattgcctgcacccttaaatggtgagatcatatgcagcgctggagttgtttaattaaaccaagattcccagaagtacatcgtattggtggttacttttgttttactaacacatgactgtaattagggggtatattactagcaacgttaatgatagatcaatagatcatgccatggagcttttatgttgtcaattgatgcctatttattatttatcattgatcatgcgtgcatttaacaggaatatgggccatactaagaaacaatgaaatgctaacttggccagagaaggtgaagtttgctcttggacttttgccagcaatggttggtggccaagcttatgttgaagctcaagatggttttactgtttctgagtggatgaaaaagcaggtataagttcacaatatcagtttgtcaagtctctgtgtacaagacacatttctacctcattaatttggaatggatataggagaaggtgttgtaagctagaaaaccttttattttctaataaaaaaactgatgccctttattgttgcattcacattgggaagaactggcagttctgaggatgaaatgcttcatgtactcaagtttatgccctttattttgcccagatccttttgcacaggtttaagcttgagctatgcttttagtttaagaccactgtttcagttaaaggtcaacaaccttgcatgatttcttcctccacctagaaaagccattgcacatattgacaaagcacacaatcctgttgactatattctttatgagctaatatacagaactgttttatacagaaaacacaatacatatgctatagttatcaatctctttccctttttttgggataacggattaatatggtgcctgatacagttgtttgatcagcacagggtgttcctgatcgagtgaacgatgaggttttcattgcaatgtcaaaggcacttaatttcataaatcctgatgagttatccatgcagtgcattctgattgctttaaaccgatttcttcaggtatttattatgttgctctatggtcatgtgtgttgcatatgagtaattcttctgttctttccggagtagtaccttacgtattacatccttcttagtgtttcttgtctctgttgtttcctaccttgaggaaactcaaatgaattttcgcttagaggccttttaaaaaaaattatgcaaatgtgtaggagaagcatggttctaagatggcattcttggatggtaatcctcctgaaaggttatgcatgcctattgttgaccatgttcgctctttgggtggtgaggttcggctgaattctcgtattcagaaaatagaacttaatcctgatggaacagtgaaacactttgcacttactgatggaactcaaataactggagatgcttatgtttttgcaacaccaggtgattttctacaatctttgtttcttctgcagttcataaattatatatatgcggctactcattttaactgactagcctgtatttagttgatatcttgaagcttcttgtacctcaagagtggaaagaaatatcttatttcaagaagctggagaagttggtgggagttcctgttataaatgttcatatatggttggttggttgaattatttggttccaagtcggaaattactcatcatcgagtttgtggttctccttatgactcatattagtatttctgttggtttgaacatttcaggtttgatagaaaactgaagaacacatatgaccaccttcttttcagcaggtgtctcttctaattcctcatcagttttgctgtcctttcactgcctcatgcatttgctctgtgctatgactggtttatgaactaaaacgatttgtattgcccaaattgggcacattctatcctgattttgtatacattcttgattaataccaaatatcatatgtcccatgtattgatcttgttcccttttctttcaggagttcacttttaagtgtttatgcggacatgtcagtaacttgcaaggtactaactaggagacattatatgttacgaaatagtaactatctgtcatgtattattgctcttgtgtatttgttcttgggtttaccatcttcaagcatcacatgatatttattttagtagctgtaacaaaaggcccaaaagtgcatgtgttacagaaggaatccagtattaattattaaacttggaaagtagatatattttatttcagattcatttaggcaacatgtcacttggctctagagtctagattttatggaccataatagctcaggaaattaaagacatggatgcctactgaacggttttctttccttttgttttgaactctttacaggaatactatgatccaaaccgttcaatgctggagttggtctttgctcctgcagaggaatgggttggacggagtgacactgaaatcatcgaagcaactatgcaagagctagccaagctatttcctgatgaaattgctgctgatcagagtaaagcaaagattctgaagtatcatgttgtgaagacaccaaggtgaggacattttgcaagagcgccccctatctgatatatcataggtaggtctaatagttggatgcacacttctctcacgttcctttcttttctgtctcactgttacagatctgtttacaagactatcccggactgtgaaccttgccgacctctgcaaagatcaccgattgaagggttctatctagctggtgactacacaaagcagaaatatttggcttcgatggagggtgcagttctatctgggaagctttgtgctcagtctgtagtggaggtaaacgctgctctccatggttctgtttgtacatagatgcatcagacttgtattgttgtcttggtgcagttcacaatgattcagttttgtaggctaatgagttatcacttgctgatttcaggattataaaatgctatctcgtaggagcctgaaaagtctgcagtctgaagttcctgttgcctcctagttgtagtcaggactattcccaatggtgtgtgtgtcatcatcccctagtcagtttttttctatttagtgggtgcccaactctccaccaatttacacatgatggaacttgaaagatgcctattttggtcttatcatatttctgtaaagttgatttgtgactgagagctgatgccgatatgccatgctggagaaaaagaacattatgtaaaacgacctgcatagtaattcttagacttttgcaaaaggcaaaaggggtaaagcgaccttttttttctatgtgaagggattaagagacctta

SEQ ID NO: 6 - DNA, 23 bp, Artificial sequence
  OsPDS_target_1
gttggtctttgctcctgcagagg

SEQ ID NO: 7 - DNA, 22 bp, Artificial sequence
  OsPDS_target_2
cctgcagaggaatgggttggac

SEQ ID NO: 8 - DNA, 23 bp, Artificial sequence
  OsPDS_target_3
cctgttataaatgttcatatatg

SEQ ID NO: 9 - DNA, 23 bp, Artificial sequence
  OsPDS_target_4
ccttacgtattacatccttctta

SEQ ID NO: 10 - DNA, 23 bp, Artificial sequence
  OsPDS_target_5
acagttgtttgatcagcacaggg

SEQ ID NO: 11 - protein, 11 aa, Artificial sequence
  Cell penetrating peptide sequence.
KKLFKKILKYL

SEQ ID NO: 12 - protein, 16 aa, Artificial sequence
  Polycation sequence
HHCRGHTVHSHHHCIR